(19) United States
(12) Patent Application Publication
(10) Pub. No.: US 2022/0136016 A1
(43) Pub. Date: May 5, 2022

(54) MODIFIED CHLORAMPHENICOL ACETYLTRANSFERASE AND BIOSYNTHESIS METHOD OF MAKING ESTERS USING SAME

(71) Applicant: UNIVERSITY OF TENNESSEE RESEARCH FOUNDATION, Knoxville, TN (US)

(72) Inventors: Cong T. TRINH, Knoxville, TN (US); Hyeongmin SEO, Elkton, MD (US); Jong-Won LEE, Knoxville, TN (US)

(73) Assignee: UNIVERSITY OF TENNESSEE RESEARCH FOUNDATION, Knoxville, TN (US)

(21) Appl. No.: 17/453,306

(22) Filed: Nov. 2, 2021

Related U.S. Application Data
(60) Provisional application No. 63/108,572, filed on Nov. 2, 2020.

(51) Int. Cl. C12P 7/62; C12N 9/10

(52) U.S. Cl. CPC C12P 7/62; C12Y 203/01028; C12N 9/1033

(57) ABSTRACT

A modified chloramphenicol acetyltransferase comprising a tyrosine residue 20 having a phenylalanine (Y20F) mutation, a microorganism harboring the modified chloramphenicol acetyltransferase, and a method of producing ester by feeding the microorganism are disclosed. The method includes providing the microorganism harboring a modified chloramphenicol acetyltransferase in an environment suitable for the microorganism to produce an ester and feeding the microorganism (i) a sugar or a cellulose, and (ii) an alcohol and/or a carboxylic acid.

### RELATED APPLICATIONS

This application claims the benefit of U.S. Provisional Application No. 63/108,572, filed Nov. 2, 2020, the entirety of which is incorporated herein by reference.

### STATEMENT REGARDING FEDERALLY SPONSORED RESEARCH

This invention was made with government support under contract 0578301-19-0023 awarded by the Department of Energy. The government has certain rights in the invention.

### REFERENCE TO SEQUENCE LISTING SUBMITTED VIA EFS-WEB

This application includes an electronically submitted sequence listing in .txt format. The .txt file contains a sequence listing entitled “05820.0025US1_ST25.txt” created on Nov. 1, 2021 and is 55,568 bytes in size. The sequence listing contained in this .txt file is part of the specification and is hereby incorporated by reference herein in its entirety.

### TECHNICAL FIELD

Modified chloramphenicol acetyltransferases (CATs) and a biosynthesis platform for production of esters using the modified chloramphenicol acetyltransferases.

### BACKGROUND

Esters are industrially important chemicals with applications as, but not limited to, flavors, fragrances, solvents, and drop-in fuels. To replace conventional petroleum-based ester synthesis, metabolic engineering and synthetic biology approaches have been pursued for at least a decade. However, harnessing metabolic capacities of various microbes for ester production is limited due to a lack of robust and efficient alcohol acyltransferases (AATs) exhibiting high compatibility with various precursor pathways and microbial hosts.

In nature, volatile esters are formulated by an alcohol acyltransferase (AAT, EC 2.3.1.84) that condenses an alcohol and an acyl-CoA in a thermodynamically favorable reaction, providing flavors and fragrances in ripening fruits and fermenting yeasts and having an ecological role in pollination. Inspired by nature, most of the metabolic engineering and synthetic biology strategies have deployed the eukaryotic AATs originating from plants or yeasts for microbial biosynthesis of target esters. However, these eukaryotic AATs lack robustness, efficiency, and compatibility as they commonly exhibit poor enzyme expression, solubility, and thermostability in microbes, thus limiting optimal microbial production of esters. In addition, limited knowledge on substrate profiles and specificities of AATs often requires laborious bioprospecting of AATs for individual target esters.

Chloramphenicol O-acetyltransferase (CAT, EC 2.3.1.28) is an antibiotic resistance enzyme that detoxifies chloramphenicol and derivative antibiotics, which inhibit protein elongation in organisms and cause cell death, by acetylation. Organisms resist this potent drug by harboring CATs that display nearly perfect catalytic efficiency at recruiting an acetyl-CoA(s) to detoxify chloramphenicol. In nature, the CAT gene is one of the most widespread genetic elements, expressing a functional enzyme in a wide range of organisms including plants, animals, and bacteria. Interestingly, when being used as antibiotic selection in a recombinant Escherichia coli, some CATs exhibit substrate promiscuity resulting in unexpected production of esters. Recently, an engineered cellulolytic thermophile Clostridium thermocellum (Hungateiclostridium thermocellum) harboring a CAT derived from a mesophile Staphylococcus aureus (CATsa) is capable of producing isobutyl acetate from cellulose at elevated temperatures.

There is a need to develop robust and efficient AATs compatible with multiple pathways and microbial hosts to expand biological routes for designer ester biosynthesis, more specifically a method having a CAT functioning as a robust and efficient AAT.

### SUMMARY

In all aspects, modified chloramphenicol acetyltransferases modified at the tyrosine residue 20 to have a phenylalanine (Y20F) mutation is described herein. In one embodiment, an additional amino acid mutation is present of a phenylalanine residue 97 to have a tryptophan (F97W) and/or an alanine residue 138 to have a threonine (A138T). In all these embodiments, the chloramphenicol acetyltransferase is from one or more of the following: Staphylococcus aureus, Escherichia coli, Haemophilus influenzae, Clostridium butyricum, and Lysinibacillus boronitolerans. A few, non-limiting examples of modified chloramphenicol acetyltransferases include CATsa Y20F, CATsa Y20F F97W, CATsa Y2OF A138T, CATsa Y20F F97W A138T, CATec3 Y20F, or CATec3 F97W Y20F.

In all embodiments, the chloramphenicol acetyltransferase can have a wild type melting temperature that is greater than 60° C. and a specific activity toward isobutanol at 50° C. of greater than 5 μmol/min/mg protein.

The modified chloramphenicol acetyltransferase can have a post-mutation melting point that is greater than 83° C. and a post-mutation specific activity toward at least isobutanol at 50° C. (k_{cat}/K_{M}) that is greater than 5 1/M/s. In one embodiment, the post-mutation specific activity is towards at least two different alcohols. The alcohols are selected from the group consisting of butanol, prenol, furfuryl alcohol, pentanol, isoamyl alcohol, benzyl alcohol, hexanol, 3-cis-hexen-1-ol, phenylethyl alcohol, 3-methyoxybenzyl alcohol, geraniol, citronellol, and nerol.

In another aspect, microorganisms harboring any one of the modified chloramphenicol acetyltransferases that have a tyrosine residue 20 with a phenylalanine (Y20F) mutation are disclosed. The microorganism can be selected from the group consisting of Clostridium acetobutylicum, Clostridium propionicum, Clostridium kluyveri, Clostridium thermocellum, Clostridium clariflavum, Clostridium celluloyticum, Clostridium beijerinckii, Clostridium tyrobutyricum, Caldicellulosiruptor bescii, Thermoanaerobacterium thermosaccharolyticum, Lactococus lactis, Bacillus subtilis, Corynebacterium glutamicum, Acidothermus cellulolyticus, Pseudomonas putida, Escherichia coli, Ralstonia eutropha, Cyanobacteria spirulina, Acinetobacter baylyi, Aspergillus niger, Aspergillus pseudoterreus, Bacillus coagulans, Clostridium autoethanogenum, Clostridium carboxidivorans, Clostridium ljungdahlii, Cupriavidus necator, Pichia kudriavzevii, Pichia pastoris, Rhodosporidium toruloides, Saccharomyces cerevisiae, Yarrowia lipolytica, Zymomonas mobilis, Geobacillus caldoxylosilyticus, Geobacillus galactosidasius, Geobacillus icigianus, Geobacillus jurassicus, Geobacillus kaustophilus, Geobacillus lituanicus, Geobacillus stearothermophilus, Geobacillus subterraneus, Geobacillus thermantarcticus, Geobacillus thermocatenulatus, Geobacillus thermodenitrificans, Geobacillus thermoglucosidasius, Geobacillus G. thermoleovorans, Geobacillus toebii, Geobacillus uzenensis, Geobacillus vulcani, Geobacillus LC300, and combinations thereof. The microorganism can be a mesophilic microorganism or a thermophilic microorganism.

In yet another aspect, methods of producing esters by feeding any one of the microorganisms harboring a modified chloramphenicol acetyltransferase described herein in an environment suitable for the microorganism to produce an ester are described. The method includes providing such a microorganism, feeding the microorganism a sugar or a cellulose, and feeding the microorganism an alcohol and/or a carboxylic acid. The method may also include extracting the ester to maintain non-toxic ester levels in the system.

In one embodiment, the method includes feeding the microorganism a mixture of alcohols to produce a plurality of esters. The mixture of alcohols can be tailored to have a preselected concentration for each alcohol to produce a preselected ester profile. The method of claim 11, comprising feeding the microorganism a carboxylic acid and/or an alcohol to produce a carboxylic acid ester.

In all aspects of the method, the feeding can occur in a fed-batch system, such as a system that includes intermittent feeding of the alcohol of greater than 10 g/L.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is schematic representation of the production of esters from an engineered microbe harboring a modified chloramphenicol acetyltransferase (CAT) having a Y20F mutation.

FIG. 2A is a phylogenetic tree of 28 wild type (natural) CATs including a heat map of their melting temperatures (Tm) and their activities towards isobutanol (IBOH).

FIG. 2B is a bar graph of the isobutanol activity values used for the phylogenetic tree of FIG. 2A.

FIG. 3 provides a vertical and horizontal view of natural CATec3.

FIG. 4 is bar graph of melting temperatures of comparative CATec3 with mutated CATec3.

FIG. 5 is a bar graph of catalytic efficiencies toward isobutanol of comparative CATec3 with mutated CATec3.

FIG. 6 is a plurality of bar graphs comparing catalytic efficiencies of natural CATec3 to CATec3 Y20F toward, from left to right, butanol, pentanol, benzyl alcohol, 2-phenylethyl alcohol, and chloramphenicol.

FIG. 7 is a bar graph of the relative activity of CATec3 Y20F towards acyl-CoAs as compared to acetyl-CoA.

FIG. 8 is a graph of melting temperatures and catalytic efficiencies toward isobutanol for natural CATsa and CATec3 compared to mutated versions of each.

FIG. 9 is a representative view of CATsa with reaction cites shown in the enlarged view.

FIG. 10 is a proposed reaction mechanism for ester biosynthesis from an alcohol and acetyl-CoA using CATsa as the representative CAT.

FIG. 11 is a bar graph of melting temperatures of comparative examples of CATsa and CATsa mutated to include Y20F.

FIG. 12 is a bar graph of catalytic efficiencies toward isobutanol of comparative examples of CATsa and CATsa mutated to include Y20F.

FIG. 13 is a representation of the amino acid residue Y20F with His-189 on CATsa and an alcohol in a transition state, having a H-bond and Van der Waals forces represented by dashed lines.

FIG. 14 is an Escherichia coli bacterium harboring CATec3 Y20F showing the route of ester production.

FIG. 15 is a Clostridium thermocellum bacterium harboring a CATec3 Y20F showing the route of ester production.

FIG. 16 is a bar graph providing alcohol conversion efficiencies for the E. coli bacterium of FIG. 14.

FIG. 17 is a bar graph of titers of ester collected from a fed-batch conversion of isoamyl alcohol and aromatic phenylethyl alcohol for the E. coli bacterium of FIG. 14.

FIG. 18 is a bar graph providing alcohol conversion efficiencies for the C. thermocellum bacterium of FIG. 15.

FIG. 19 is a bar graph of titers of ester collected from a fed-batch conversion of isobutanol for the C. thermocellum bacterium of FIG. 15.

FIG. 20 is a bar graph of the g/L of a synthesized rose profile generated be feeding the E. coli bacterium of FIG. 14 the seven alcohols that result in the seven esters of the rose profile.

FIG. 21 is a combined bar graph and chart of recombinant C. thermocellum harboring various CATs with distinctive melting temperatures and catalytic efficiency for in vivo isobutyl ester production by co-feeding cellulose and isobutanol at 55° C. thereto.

FIG. 22 is a bar graph of the effect of temperature on isobutyl ester production of a C. thermocellum bacterium harboring CATsa Y20F A138T.

FIG. 23 is a bar graph of the effect of temperature on isobutyl ester production of a C. thermocellum bacterium harboring CATec3 Y20F.

FIG. 24 is a reaction scheme for the production of isoamyl lactate from an E. coli harboring CATec3 Y20F.

FIG. 25 is a bar graph of the effect of expressing various PCTs on isoamyl lactate production after 48 hours.

FIG. 26A is the first half of a chart of esters that can be made by the modified bacterium discussed herein.

FIG. 26B is the second half of the chart of esters that can be made by the modified bacterium discussed herein.

FIG. 27 is a chart of designer esters made by modified bacterium that are fed sugars or cellulosic biomass.

FIG. 28 is photograph of degradation of Avicel PH-10 samples over a period of 144 h.

FIG. 29 is photograph of degradation of popular-CELF-pretreated cellulose samples over a period of 144 h.

FIG. 30 is graph of the ester production at 144 hours for samples shown in FIGS. 28 and 29.

### DETAILED DESCRIPTION

The following detailed description will illustrate the general principles of the invention, examples of which are additionally illustrated in the accompanying drawings. In the drawings, like reference numbers indicate identical or functionally similar elements.

Except in the working examples, or where otherwise explicitly indicated, all numbers in this description indicating amounts, parts, percentages, ratios, and proportions of material, physical properties of material, and conditions of reaction are to be understood as modified by the word “about.” “About” as used herein means that a value is preferably +/−5% or more preferably +/−2%. Percentages for concentrations are typically % by wt.

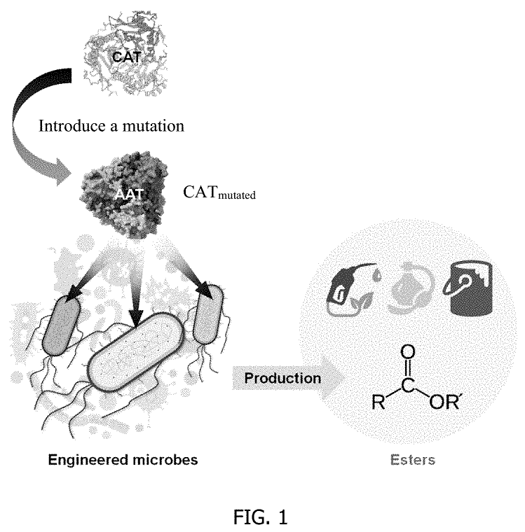

Turning to FIG. 1, a schematic illustration is provided showing a CAT that is mutated to form a mutated CAT that can function like an alcohol acyltransferase, which is then introduced into a microbe, such as a bacterium, yeast, or fungus to form an engineered microbe that can be fed sugars or cellulose and an alcohol and/or a carboxylic acid to produce an ester. The mutated CAT has a tyrosine residue 20 having a phenylalanine (Y20F) mutation. The mutated CAT can have an additional amino acid mutation selected from the group consisting of a phenylalanine residue 97 having a tryptophan (F97W), and/or an alanine residue 138 having a threonine (A138T). Some CATs evaluated and studied herein do not include a homolog residue of Ala-138 such that the A138T mutation is not possible therein. CATec3 discussed herein does not have a homolog residue of Ala-138.

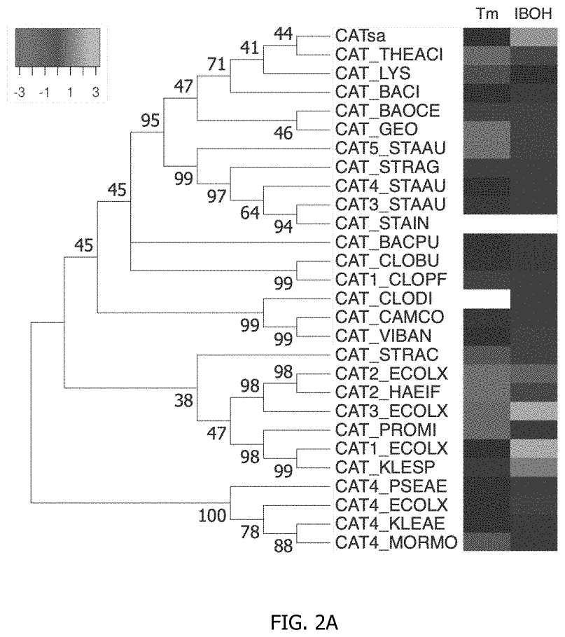

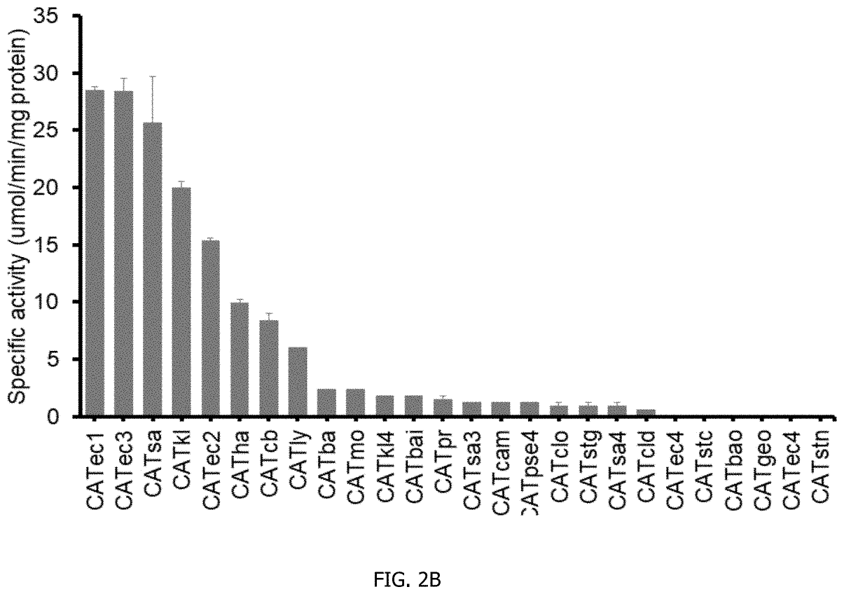

Referring to FIG. 2A, the phylogenetic tree of the wild type 28 CATs (provided as SEQ ID NOS: 1-28, which are expressly incorporated herein), representing both type A and type B that are structurally distinct, was built based on aligned sequences performed using MEGA7 based on the maximum likelihood method with 1,000 bootstrap replicates. A 40% bootstrap confidence level cutoff was selected. The phylogenetic tree represents the high thermostability and alcohol promiscuity of the CATs in nature (i.e., wild type). The 28 CATs were synthesized, expressed, purified, and characterized for their melting temperatures (Tm) and promiscuous activities towards isobutanol (IBOH). Most of the CATs showed melting temperatures higher than 60° C. except CAT_GEO (Tₘ=43.5° C.).

Among the CATs, eight exhibited the highest specific activities towards isobutanol at 50° C.: (1) CAT1_ECOLIX (CATec1), (2) CAT3_ECOLIX (CATec3), (3) CATsa, (4) CAT_KLEPS (CATk1), (5) CAT2_ECOLIX (CATec2), (6) CAT_HAEIF (CATha), (7) CAT_LYS (CAT1y) and (8) CAT_CLOBU (CATcb). Here, “sa” stands for Staphylococcus aureus, “ec” stands for Escherichia coli, “ha” stands for Haemophilus influenzae, “cb” stands for Clostridium butyricum, and “kl” stands for Klebsiella sp. and “lys” stands for Lysinibacillus boronitolerans. With reference to FIG. 2B, the selected threshold for specific activity toward 100 mM isobutanol at 50° C. was greater than 5 μmol/min/mg protein. Five out of these eight most isobutanol-active CATs were evolutionarily related, suggesting that their activities towards isobutanol might be influenced by their unique structural features.

Accordingly, in all example embodiments provided is a modified CAT protein having a Y20F amino acid substitution mutation. As used herein, “amino acid” or “amino acid residue” or “residue” refers to any naturally occurring amino acid, any non-naturally occurring amino acid, any modified including derivatized amino acid, or any amino acid mimetic known in the art. The amino acid may be referred by both their common three-letter abbreviation and single letter abbreviation. In certain example embodiments, the modified CAT proteins can be about 80%, 85%, 90%, 95%, 98% or more sequence identity to any one of SEQ ID NOS: 1-28, wherein the CAT protein includes a Y20F mutation. That is, the CAT modified protein, although having an amino acid sequence at least partially or fully identical to any one of SEQ ID NOS: 1-28, retains a Y20F amino acid substitution. In certain example embodiments, the modified CAT protein is a functional fragment of any one of SEQ ID NOS: 1-28, the sequence of the fragment corresponding to one or more regions of the otherwise full-length amino acid sequences while retaining the Y20F substitution, as described herein. In certain example embodiments, the functional fragment including the Y20F substitution has 80%, 85%, 90%, 95%, 98% or more sequence identity to one or more regions of the full-length sequence set forth as any one of SEQ ID NOS: 1-28. In certain example embodiments, each of the amino acids of SEQ ID NOS:1-28 can be L-form amino acids. In certain example embodiments, one or more of the amino acids forming all or a part of the modified CAT proteins or functional fragments thereof can be stereoisomers. That is, any one or more of the amino acids of the modified CAT protein or functional fragments thereof can be a D- or L- amino acid. And in certain example embodiments, the modified CAT proteins or functional fragments thereof can also include one or more modified amino acids. The modified amino acid may be a derivatized amino acid or a modified and unusual amino acid. Examples of modified and unusual amino acids include but are not limited to, 2-Aminoadipic acid (Aad), 3-Aminoadipic acid (Baad), β-Amino-propionic acid (Bala, β-alanine), 2-Aminobutyric acid (Abu, piperidinic acid), 4-Aminobutyric acid (4Abu), 6-Aminocaproic acid (Acp), 2-Aminoheptanoic acid (Ahe), 2-Aminoisobutyric acid (Aib), 3-Aminoisobutyric acid (Baib), 2-Aminopimelic acid (Apm), 2,4-Diaminobutyric acid (Dbu), Desmosine (Des), 2,2′-Diaminopimelic acid (Dpm), 2,3-Diaminopropionic acid (Dpr), N-Ethylglycine (EtGly), N-Ethylasparagine (EtAsn), Hydroxylysine (Hyl), allo-Hydroxylysine (Ahyl), 3-Hydroxyproline (3Hyp), 4-Hydroxyproline (4Hyp), Isodesmosine (Ide), allo-Isoleucine (Alle), N-Methylglycine (MeGly, sarcosine), N-Methylisoleucine (Melle), 6-N-Methyllysine (MeLys), N-Methylvaline (MeVal), Norvaline (Nva), Norleucine (Nle), and Ornithine (Orn). Other examples of modified and unusual amino acids are described generally in Synthetic Peptides: A User's Guide, Second Edition, April 2002, Edited Gregory A. Grant, Oxford University Press; Hruby V J, Al-obeidi F and Kazmierski W: Biochem J268:249-262, 1990; and Toniolo C: Int J Peptide Protein Res 35:287-300, 1990; the teachings of all of which are expressly incorporated herein by reference. In certain example embodiments, the modified CAT protein or functional fragments thereof can be detectably labeled with a known label, such as a fluorescent or radioactive label.

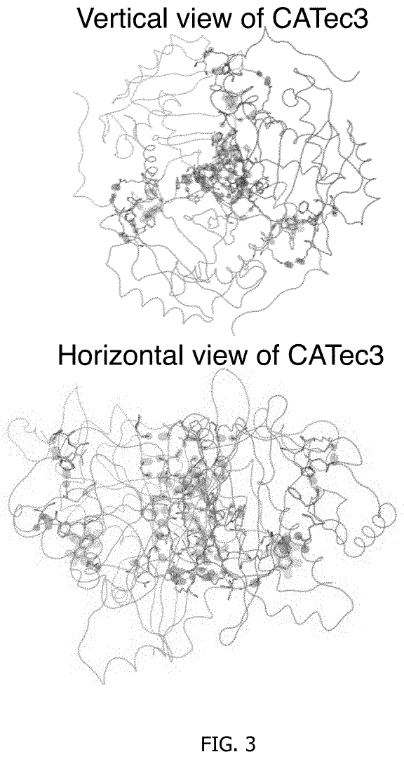

Then, the kinetic thermostability thereof were evaluated by measuring their activity losses after one-hour incubation at elevated temperatures of 50° C., 55° C. 60° C., 65° C., and 70° C. to select the most promising candidates. Remarkably, CATec3 and CATec2, derived from a mesophilic E. coli, retained more than 95% of the activity at 70° C., which is why CATec3 was selected for the majority of the tests conducted and reported herein. As shown in FIG. 3, visualized contacts in CATec3, which includes hydrogen bonds, metal, ionic, arene, covalent, and Van der Waals distance were analyzed in the structure. For FIG. 3, the Swiss-Model and ‘Builder’ tools of a commercial Molecular Operating Environment (MOE) software, version 2019.01 was used to generate the three-dimensional (3D) structures.

CATsa F97W was previously disclosed by Applicant and was used for a comparative example against CATs mutated to have the Y20F substitution.

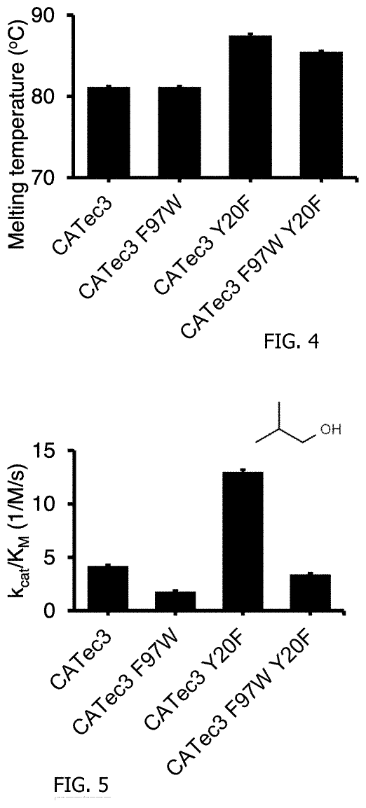

Turning now to FIGS. 4 and 5, the CATec3 having the Y20F mutation alone (87.5° C.) or in combination with F97W (85.5° C). have a post-mutation melting point that is greater than 83° C. and, preferably, a post-mutation specific activity toward isobutanol at 50° C. (k_{cat}/K_{M}) that is greater than 5 1/M/s. In FIG. 4, the melting point of the wild type CATec3 was 80.2° C. and CATec3 F97W was 81.2° C.

Turning now to FIG. 6, the CAT have the Y20F has a post-mutation specific activity at 50° C. (k_{cat}/K_{M}) that is greater than 5 1/M/s toward at least two different alcohols selected from the group consisting of butanol, prenol, furfuryl alcohol, pentanol, isoamyl alcohol, benzyl alcohol, hexanol, 3-cis-hexen-1-ol, phenylethyl alcohol, 3-methyoxybenzyl alcohol, geraniol, citronellol, and nerol, and more preferably toward three, four, or five or more of said alcohols. As the graphs in FIG. 6 show, CATec3 Y20F was more efficient towards bulky and long-chain alcohols that are more hydrophobic than short-chain alcohols, likely due to a stronger binding affinity. As compared to the wild type CATec3 against six alcohols selected as a representative set of those that can be naturally synthesized by organisms, we found that the CATec3 Y20F variant exhibited much higher catalytic efficiency towards butanol by 4.0-fold, pentanol by 8.8-fold, benzyl alcohol by 6.9-fold, and phenylethyl alcohol by 6.2-fold. In contrast, the catalytic activity of CATec3 Y20F towards the native substrate chloramphenicol decreased about 3.2-fold as compared to the wild type activity.

In addition, acetylation of fatty alcohols such as octanol and decanol to produce long-chain esters that can potentially be used for drop-in biodiesel applications was possible using CATec3 Y20F. The alcohol compatibility of CATec3 Y20F expanded from ethanol to terpenoid alcohols such as geraniol and nerol. Due to high K_{M} value (>1M) towards ethanol, CATec3 Y20F is more favorably applied for biosynthesis of higher-chain alcohol esters. This characteristic is potentially beneficial to produce designer esters rather than ethyl esters in organisms since ethanol is a common fermentative byproduct that can act as a competitive substrate. In comparison to CATsa Y2OF A138T, CATec3 Y20F displayed higher activity towards not only isobutanol, but also most of other alcohols. It is noteworthy that these engineered CATs exhibited different alcohol specificities. For example, CATsa Y20F A138T was relatively more specific to phenylethyl alcohol than terpenoid alcohols as compared to CATec3 Y20F.

The results showed that the CATec3 Y20F improved not only the catalytic efficiency (13.0±0.2, 1/M/s), about 3.3-fold higher than its wild type, but also the melting temperature increased (87.5±0.5° C.). Among all the CATs characterized, CATec3 Y20F is the most thermostable and isobutanol-active.

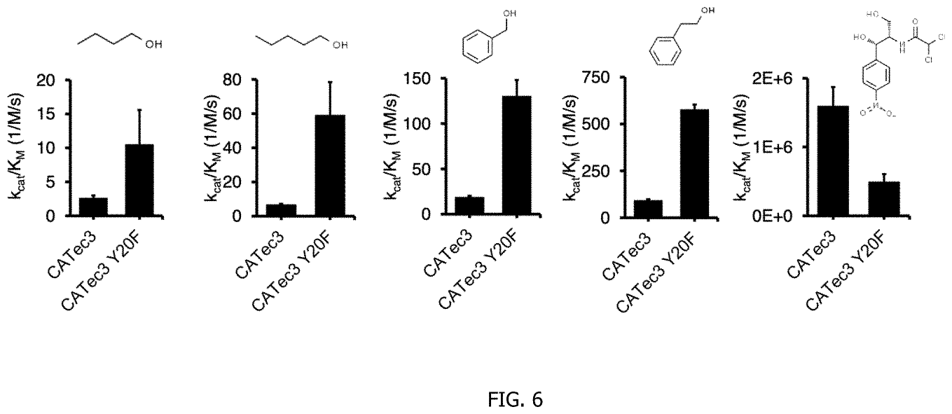

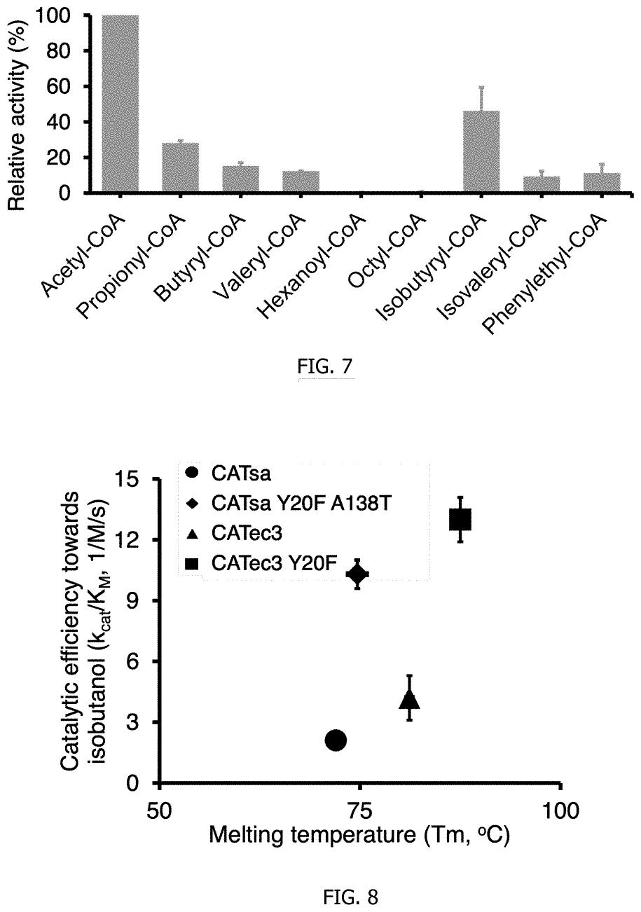

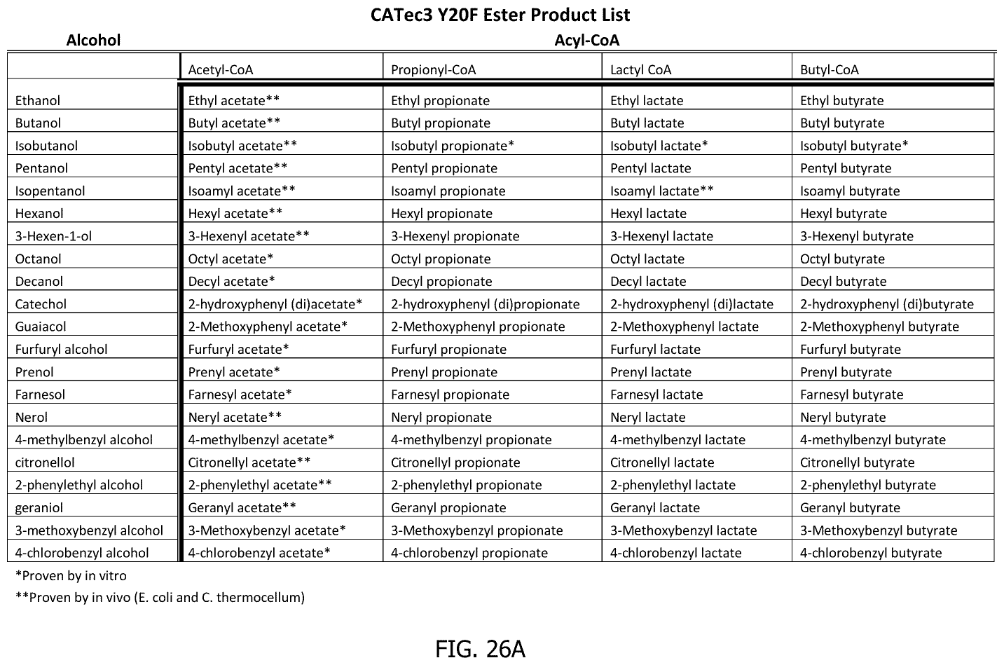

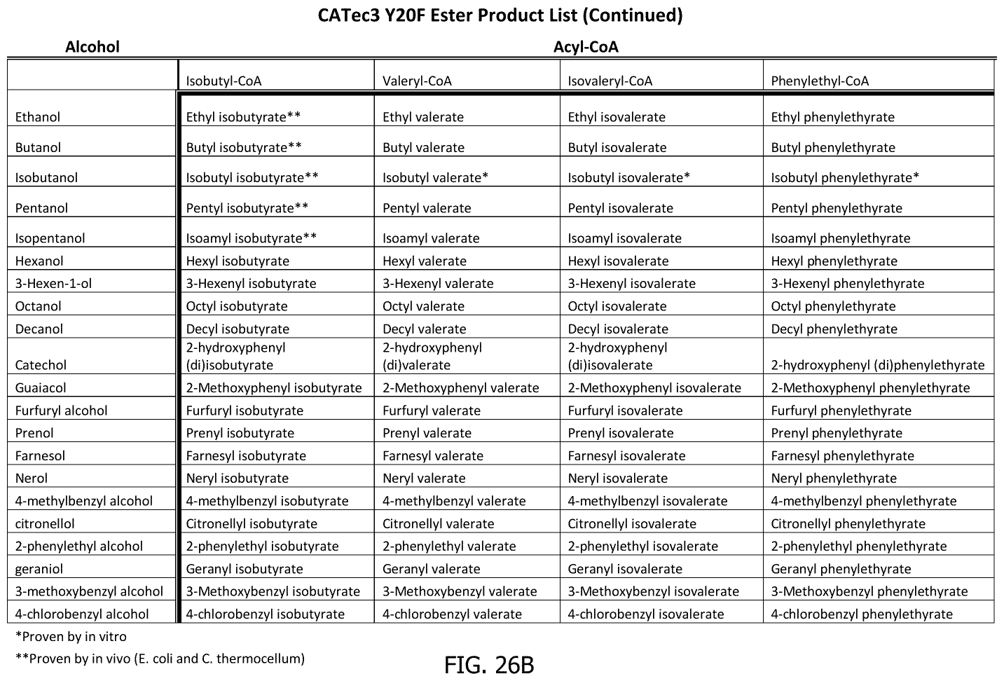

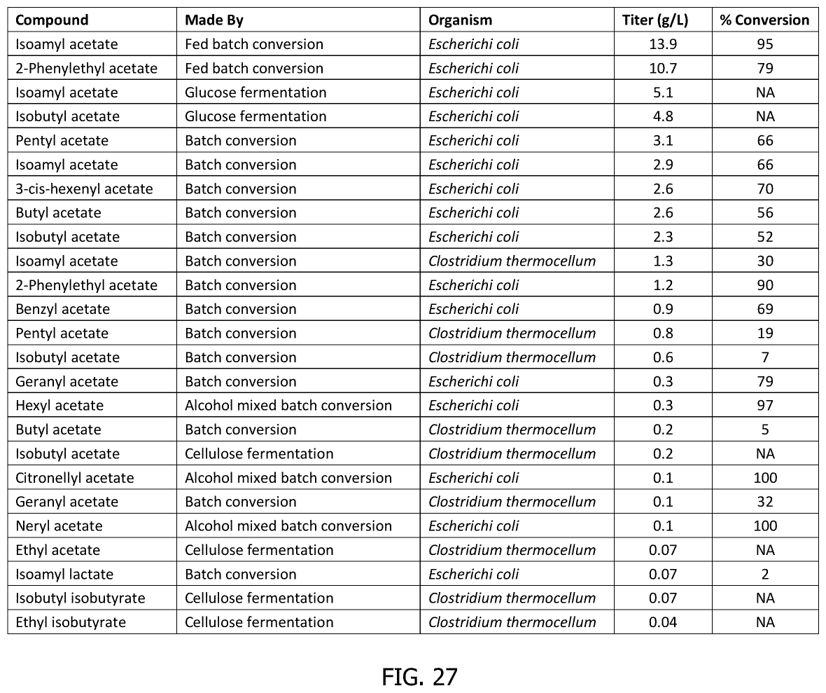

Turning now to FIG. 7, CATec3 Y20F is also compatible with longer-chain acyl-CoAs. The relative activities of CATec3 Y20F against a set of 10 linear, branched, and aromatic acyl-CoAs that can be synthesized in organisms together with isobutanol as a co-substrate were tested. CATec3 Y20F has the highest activity towards the native substrate acetyl-CoA, which is the most abundant and critical precursor metabolite for cell biosynthesis. As compared to acetyl-CoA, CATec3 Y20F achieved 46%, 28%, 15%, 12%, 11%, and 9% of activity towards isobutyl-CoA, propionyl-CoA, butyryl-CoA, valeryl-CoA, phenylethyl-CoA, and isovaleryl-CoA, respectively. These CoA components will produce acetate esters, propionate esters, butyrate esters, valerate esters, phenylethyrate esters, isovalerate esters, respectively. No activity was detected against linear fatty acyl-CoAs longer than valeryl-CoA. A chart of esters that can be made based on the alcohol fed to the bacterium and the above list of a linear fatty acyl-CoAs is provided as FIGS. 26A and 26B.

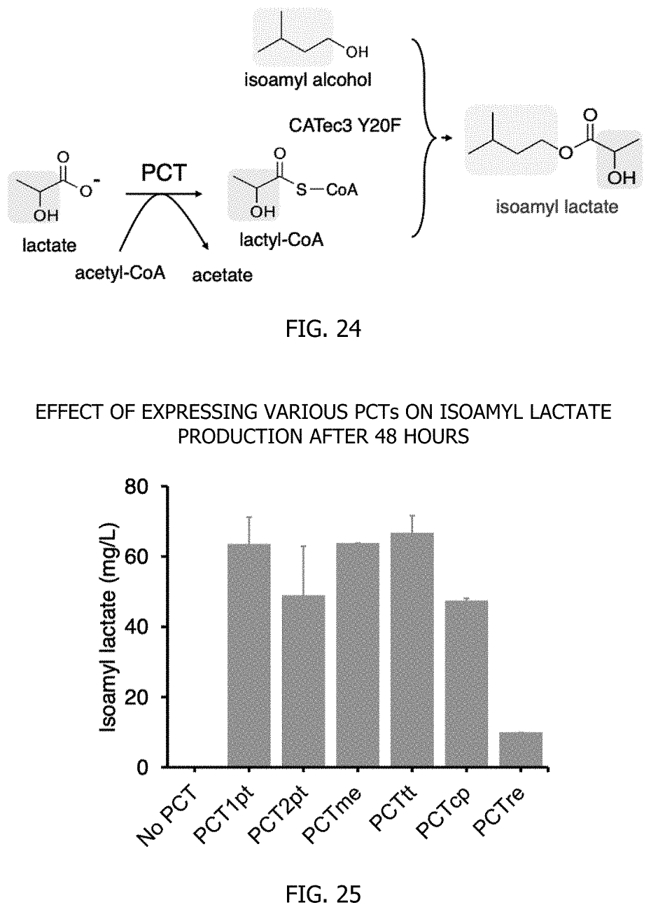

Interestingly, CATec3 Y20F also exhibited activity towards an uncommon lactyl-CoA for lactate ester biosynthesis. Since lactyl-CoA is not commercially available for in vitro assay, the activity was determined in vivo by using a recombinant E. coli co-expressing CATec3 Y20F and a propionyl-CoA transferase (PCT) derived from different microbes including Thermus thermophilus (PCTtt) that transfers CoA from acetyl-CoA to lactate. By co-feeding the recombinant E. coli with isoamyl alcohol and lactate, isoamyl lactate could be produced. A reaction scheme is provided in FIG. 24. In one example, 2 g/L each of isoamyl alcohol and lactate were fed to the recombinant E. coli with glucose to produce about 66.6 mg/L of isoamyl lactate, which is at least 2.5-fold higher than the use of the eukaryotic AATs known in the art. The initial medium contained 10 g/L glucose, 5 g/L yeast extract and 0.1 mM of IPTG, to which the isoamyl alcohol and the lactate were added. 1 mL of hexadecane was overlaid to extract the isoamyl lactate produced during fermentation. Since PCTtt is derived from a thermophile, the lactate ester biosynthesis pathway is likely robust and compatible with thermophilic hosts. Thus, CATs can be repurposed for de novo thermostable AATs. CATec3 Y20F exhibits extraordinary robustness, efficiency, and compatibility with various alcohols and acyl-CoA moieties, making it an ideal platform to synthesize designer bioesters in multiple organisms.

In additional trials, other PCTs were tested. Example PCTs include, but are not limited to, PCTpt, Pelotomaculum thermopropionicum; PCTme, Megasphera elsdenii; PCTtt, Thermus thermophilus, PCTcp, Clostridium propionicum; PCTre, Ralstonia eutropha. These PCTs were tested under the same parameters noted in the preceding paragraph for PCTtt, and the results are provided in FIG. 25. All but PCTre yielded more than 40 mg/L of isoamyl lactate. PCT1pt and PCT2pt are two copies of PCT from PCT pt. Notably, PCT1pt, PCTme, and PCTtt each yielded more than 60 mg/L of isoamyl lactate.

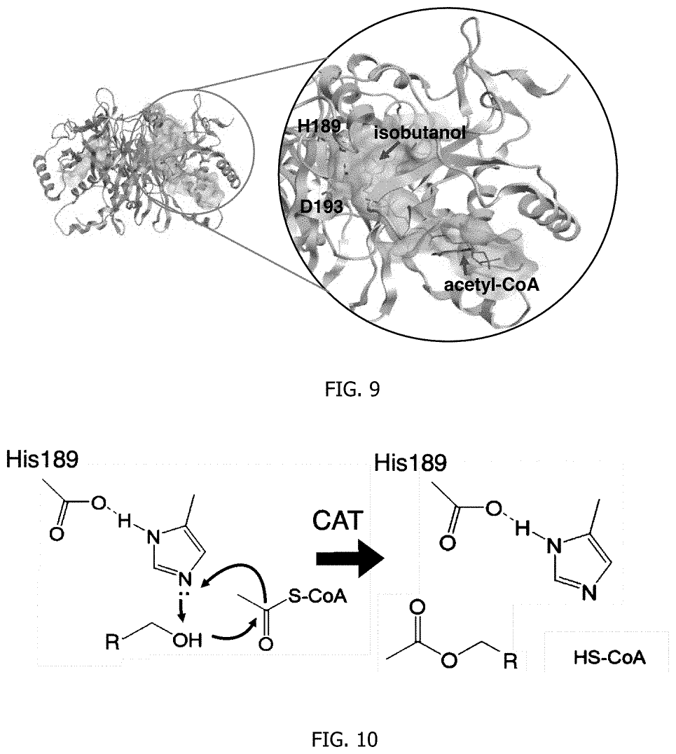

Turning now to FIG. 9, a representative view of CATsa with reaction sites shown in the enlarged view is provided. His-189 is a reaction site in the binding pocket of CATsa that binds to isobutanol, see the reaction scheme presented in FIG. 10. Using in vivo microbial screening assays known in the art, the Y20F variant was determined to exhibit a significant increase in conversion of isobutanol to isobutyl acetate, about a 43-fold increase as compared to the wild type CATsa.

The binding pocket was analyzed using the MOE software with the ‘Site Finder’ tool and selecting the best-scored site that is consistent with the reported catalytic sites. Then, docking simulations for acyl-CoA and alcohol with CATs were performed using the induced fit protocol with the Triangle Matcher placement method and the London ΔG scoring function. The best-scored binding pose exhibiting the interaction between the residue and the substrate at root-mean-square-deviation (RMSD)<2.3 Å was selected. The ‘alanine scan’ and ‘residue scan’ tools of MOE were used to identify the potential residue candidates for mutagenesis of the acyl-CoA-alcohol-CAT complex, based on the ΔStability and/or ΔAffinity values calculated. Mutant candidates with small values of the ΔStability and/or ΔAffinity are chosen for experimental testing. To perform the protein contact analysis, we used the ‘Protein Contacts’ tool of MOE.

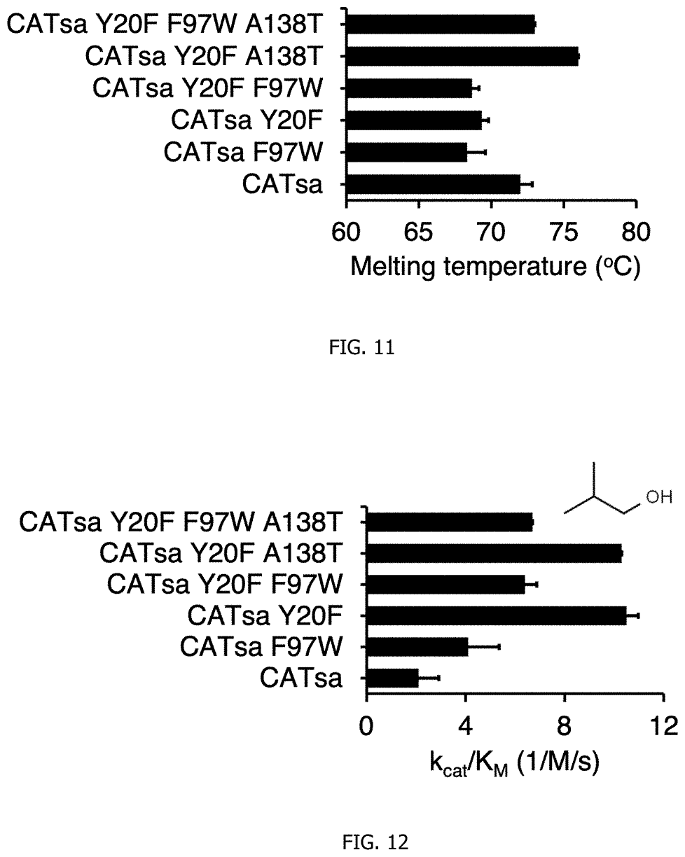

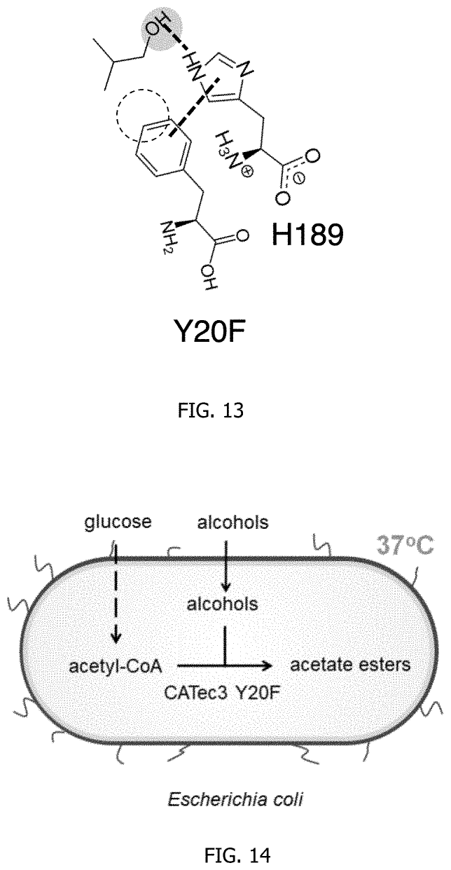

Since previous studies demonstrated that CATsa F97W improved the activity towards isobutanol and CATsa A138T increased thermostability, these mutations were included as combinatorial mutagenesis with CATsa Y20F to evaluate their effect on enzyme performance. Turning now to FIGS. 11 and 12, CATsa Y20F improved the catalytic efficiency over the wildtype CATsa and CATsa F97W by 5.0- and 2.5-fold, respectively (FIG. 12), while the melting temperature slightly decreased from 71.2 to 69.3° C. (FIG. 11). Among the combinatorial mutagenesis, CATsa Y20F A138T exhibited both the highest melting temperature (76±0.0° C.) and catalytic efficiency towards isobutanol (10.3±1.2, 1/M/s), respectively. Theoretically, it is believed that the hydrogen bonds between chloramphenicol, the catalytic site His-189, and Tyr-20 at a transition state are critical for high catalytic efficiency of the CATsa. Since the Y20F mutation retains the aromatic ring that contributes to tautomeric stabilization of His-189, the catalytic imidazole can still interact with the smaller chain alcohols flexibly as shown in FIG. 13, likely contributing to the enhanced enzymatic activity observed for isobutyl acetate biosynthesis in FIG. 12.

In one aspect, the modified CATs can be CATsa Y20F, CATsa Y20F F97W, CATsa Y20F A138T, CATsa Y20F F97W A138T, CATec3 Y20F, or CATec3 F97W Y20F.

Referring now to FIGS. 14-19, microorganisms harboring any of the modified chloramphenicol acetyltransferase having the Y20F mutation discussed herein are disclosed. The microorganisms can be yeast, fungi, or bacteria. The microorganism can be, but is not limited to, Clostridium acetobutylicum, Clostridium propionicum, Clostridium kluyveri, Clostridium thermocellum, Clostridium clariflavum, Clostridium celluloyticum, Clostridium beijerinckii, Clostridium tyrobutyricum, Caldicellulosiruptor bescii, Thermoanaerobacterium thermosaccharolyticum, Lactococus lactis, Bacillus subtilis, Corynebacterium glutamicum, Acidothermus cellulolyticus, Pseudomonas putida, Escherichia coli, Ralstonia eutropha, Cyanobacteria spirulina, Acinetobacter baylyi, Aspergillus niger, Aspergillus pseudoterreus, Bacillus coagulans, Clostridium autoethanogenum, Clostridium carboxidivorans, Clostridium ljungdahlii, Cupriavidus necator, Pichia kudriavzevii, Pichia pastoris, Rhodosporidium toruloides, Saccharomyces cerevisiae, Yarrowia lipolytica, Zymomonas mobilis, Geobacillus caldoxylosilyticus, Geobacillus galactosidasius, Geobacillus icigianus, Geobacillus jurassicus, Geobacillus kaustophilus, Geobacillus lituanicus, Geobacillus stearothermophilus, Geobacillus subterraneus, Geobacillus thermantarcticus, Geobacillus thermocatenulatus, Geobacillus thermodenitrificans, Geobacillus thermoglucosidasius, Geobacillus G. thermoleovorans, Geobacillus toebii, Geobacillus uzenensis, Geobacillus vulcani, Geobacillus LC300, and combinations thereof.

Referring now to FIG. 14, for biosynthesis of esters such as acetate esters, we engineered HSEC01, a recombinant E. coli BL21 (DE3) harboring CATec3 Y20F. The environment was maintained at 37° C. The E. coli uses its native metabolism to convert fermentable sugars into the precursor acetyl-CoA, and is fed an alcohol such that the bacterium will produce esters. The alcohol can be a linear, branched, saturated, unsaturated, and/or aromatic alcohols. The esters can be extracted upon production, for example with a hexadecane extraction. As shown in FIG. 16, in batch cultures, conversion of various alcohols to their respective acetate esters achieved more than 50% (mol/mol) yield within 24 hours. Here, such alcohol was supplemented in the medium of the recombinant E. coli at 3 g/L for n-butanol, isobutanol, n-pentanol, and isoamyl alcohol, 1 g/L for benzyl alcohol and phenylethyl alcohol, and 0.3 g/L for geraniol. Noticeably, yields of phenylethyl and geranyl acetate were greater than 80% (mol/mol). The recombinant E. coli produced and secreted bioesters at final titers of 2.6 g/L butyl acetate, 2.3 g/L of isobutyl acetate, 3.1 g/L pentyl acetate, 2.9 g/L isoamyl acetate, 2.6 g/L 3-hexenyl acetate, 0.9 g/L benzyl acetate, 1.2 g/L 2-phenylethyl acetate, and 0.3 g/L geranyl acetate.

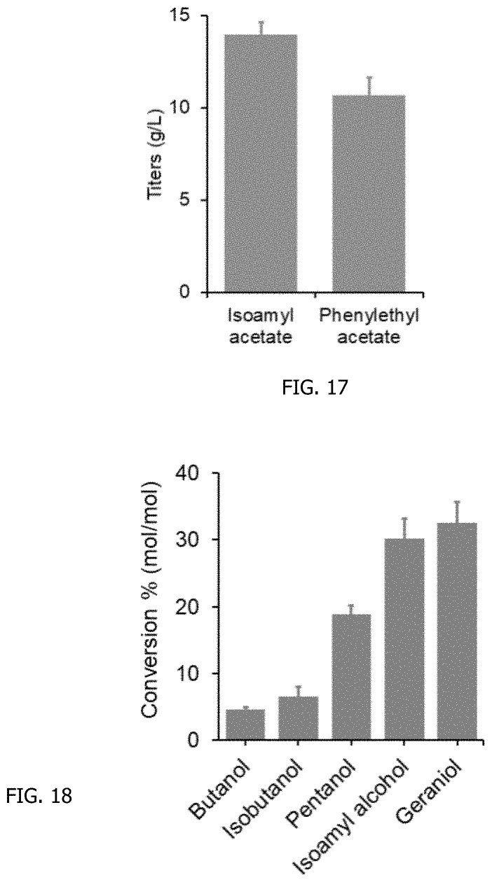

To further increase ester production, fed-batch fermentation was used for branched isoamyl alcohol and aromatic phenylethyl alcohol. With the intermittent feeding of 10 g/L alcohols as a demonstration, the recombinant E. coli produced the expected esters at a relatively high efficiency, achieving titers of 13.9 g/L and 10.7 g/L as shown in FIG. 17 and yields of 95% (mol/mol) and 80% (mol/mol) for isoamyl acetate and phenylethyl acetate, respectively. Surprisingly, even though both alcohols and esters are known to be toxic to microbial health at relatively low concentrations (<2 g/L), no noticeable growth inhibition was observed at this high level of ester production in the fed batch mode because in situ ester extraction with hexadecane was performed during the process. These results demonstrate that the recombinant CATec3 Y20E-expressing E. coli could produce all of the expected acetate esters with relatively high efficiency and compatibility.

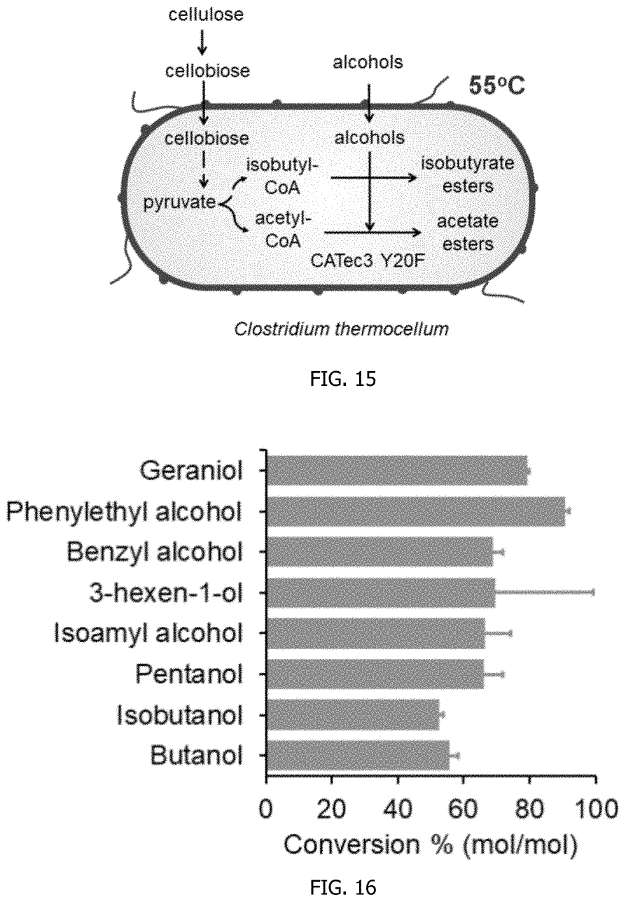

Referring now to FIG. 15, for biosynthesis of esters, we engineered C. thermocellum, a cellulolytic, thermophilic, obligate anaerobic, gram-positive bacterium, to harbor CATec3 Y20F. C. thermocellum has a native metabolic capability for effectively degrading recalcitrant cellulosic biomass at elevated temperatures (≥50° C., more preferably about 55° C.) in a single step to produce desirable chemicals, i.e., esters/bioesters. An engineered C. thermocellum Δclo1313_0613, Δclo1313_0693 was selected as the host because its two carbohydrate esterases were disrupted to alleviate ester degradation. By co-feeding cellulose and each higher alcohol, the recombinant C. thermocellum could produce all respective acetate esters (FIG. 18). For the results in FIG. 18, each alcohol was fed in to the medium at 3 g/L n-butanol, 3 g/L isobutanol, 3 g/L n-pentanol, 3 g/L isoamyl alcohol, and 0.3 g/L of geraniol.

Since C. thermocellum has the endogenous isobutyl-CoA pathway, the production of isobutyrate esters such as butyl isobutyrate and isobutyl isobutyrate as byproducts was observed. Many of these esters, such as n-butyl, n-pentyl, isoamyl, and geranyl esters, have never been reported to be feasibly synthesized in a thermophile. Among the esters, isoamyl acetate was produced at the highest conversion yield of >30% (mol/mol) and titer of 1.2 g/L.

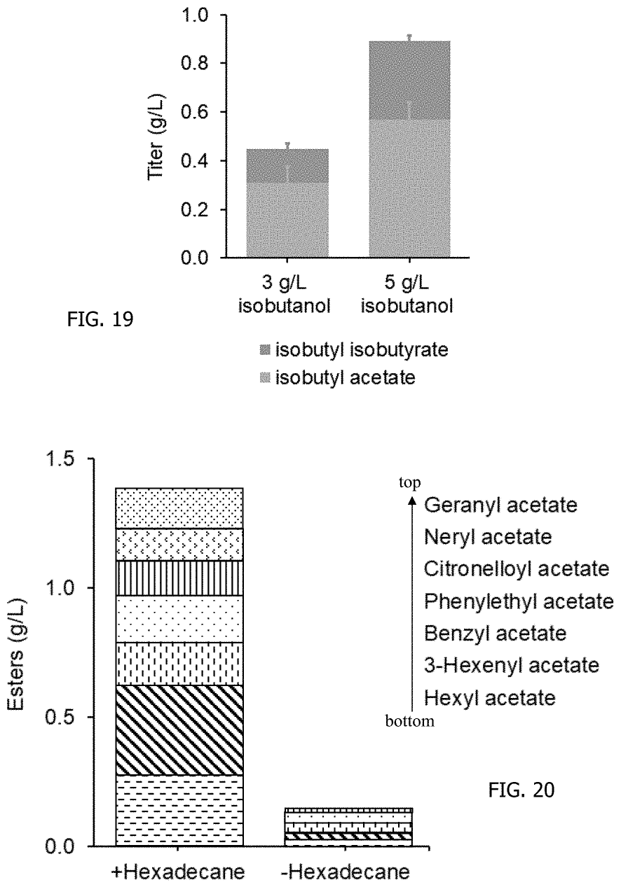

Ester production in C. thermocellum was not as high as observed in E. coli likely due to the metabolic burden required to make cellulolytic enzymes for cellulose degradation along with overexpression of the heterologous gene. Turning now to FIG. 19, an increased titer of isobutyl esters was achieved when feeding a higher concentration of isobutanol, but below a lethal concentration, indicating that the enzyme expression and/or alcohol availability were likely unsaturated in C. thermocellum.

In all aspects, methods of producing esters utilizing any of the microorganisms harboring any of the modified chloramphenicol acetyltransferase having a Y20F mutation in an environment suitable for ester production is encompassed herein. The microorganisms can be mesophilic or thermophilic, which determines the environment that is suitable for ester production. The method includes providing such a microorganism in a suitable environment, feeding the microorganism a sugar or a cellulose and an alcohol and/or a carboxylic acid. The method may also include extracting the ester to maintain non-toxic ester levels in the system, which provides the benefit of avoiding microorganism inhibition. The extraction of the esters can be an in situ extraction, such as one that uses hexadecane.

The feeding of any substance to a selected microorganism can include a mixture of sugars, a mixture of alcohols, a mixture of cellulosic materials, a mixture of carboxylic acids, and blends of any such mixtures to produce a plurality of esters. The mixtures, especially of the alcohols and/or carboxylic acids are preselected and have a preselected concentration for each alcohol or carboxylic acid to produce a preselected ester profile. See the example for a rose presented in working example 2. When carboxylic acids are fed to the cells, they will be converted to acyl CoAs by the enzyme propionyl-CoA transferase (PCT). Some example carboxylic acids include, but are not limited to, acetic acid, propionic acid, lactic acid, butyric acid, isobutyric acid, valeric acid, isovaleric acid, and hexanoic acid. The feeding can include a fed-batch system, which can utilize intermittent feedings. The feeding of the alcohol(s) and/or carboxylic acid(s) in the fed-batch system can be greater than 10 g/L.

### Working Example 1: Create an Ester Profile (Mixture of Esters)

Referring to FIG. 20, utilizing the recombinant E. coli HSEC01 harboring CATec3 Y20F, a test was conducted to mimic an ester profile of a rose, in particular Rosa hybrida's profile from the developing stage which attracts pollinators. The roses' ester profile includes geranyl acetate, neryl acetate, citronelloyl acetate, phenylethyle acetate, benzyl acetate, 3-hexenyl acetate, and hexyl acetate. To mimic this particular ester profile, the recombinant E. coli was fed a mixture of alcohols at a total working concentration of 1 g/L, consisting of 0.2 g/L hexanol, 0.2 g/L 0.15 g/L 3-cis-hexen-1-ol, benzyl alcohol, 0.15 g/L phenylethyl alcohol, 0.1 g/L geraniol, 0.1 g/L nerol, and 0.1 g/L citronellol at a mid-log phase (OD₆₀₀ₙₘ˜1.0) using a fed-batch system. The recombinant E. coli rapidly and completely converted the alcohol mixture into the desirable acetate ester profile with a yield of 97.1±0.7% (mol/mol) and a titer of ˜1.5 g/L within 12 h as shown in FIG. 20. Also, FIG. 20 compares the ester production with an in situ extraction using hexadecane (+hexadecane) to mitigate the toxicity of the esters versus without (-hexadecane). The difference in yield is significant.

### Working Example 2: Thermostability

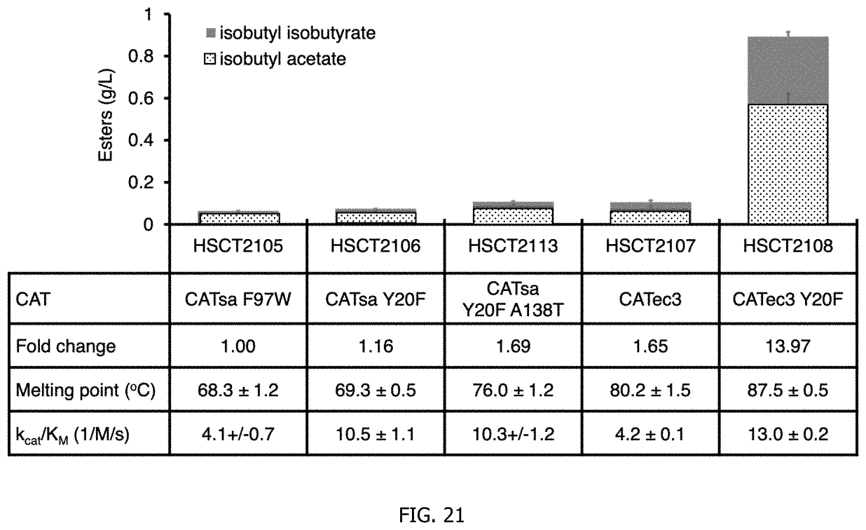

Functional expression of a heterologous protein in thermophiles requires high thermostability. Inspired by the differences in the catalytic efficiency and melting temperatures among the modified CATs, we investigated how thermostability of the CATs affected ester production in C. thermocellum. Referring to FIG. 21, we characterized the recombinant C. thermocellum Δclo1313_0613 Δclo1313_0693 harboring various CATs with distinctive melting temperatures and catalytic efficiency for in vivo isobutyl ester production by co-feeding cellulose and isobutanol at 55° C. Among the recombinant C. thermocellum strains, HSCT2108 harboring CATec3 Y20F, which has the highest catalytic efficiency and melting temperature, produced the highest level of isobutyl esters (892 mg/L), about 14-fold higher than the CATsa F97W expressing C. thermocellum HSCT2105 (a comparative example). Even though CATsa Y20F A138T has similar catalytic efficiency but higher melting temperature relative to CATsa Y20F, the CATsa Y20F A138T-expressing strain HSCT2113 produced 46% more esters than the CATsa Y20E-expressing strain HSCT2106. Similarly, we also observed higher ester production in the CATec3-expressing strain HSCT2107 (wild-type comparative example) than the CATsa F97W-expressing strain HSCT2105, where CATec3 has similar catalytic efficiency but higher melting temperature. Remarkably, both HSCT2107 and HSCT2113 produced esters at very similar level, although CATsa Y20F A138T has higher catalytic efficiency but about 10° C. lower melting temperature than CATec3. These results strongly suggested that CAT robustness with enhanced thermostability plays a critical role for efficient ester production in C. thermocellum at elevated temperatures.

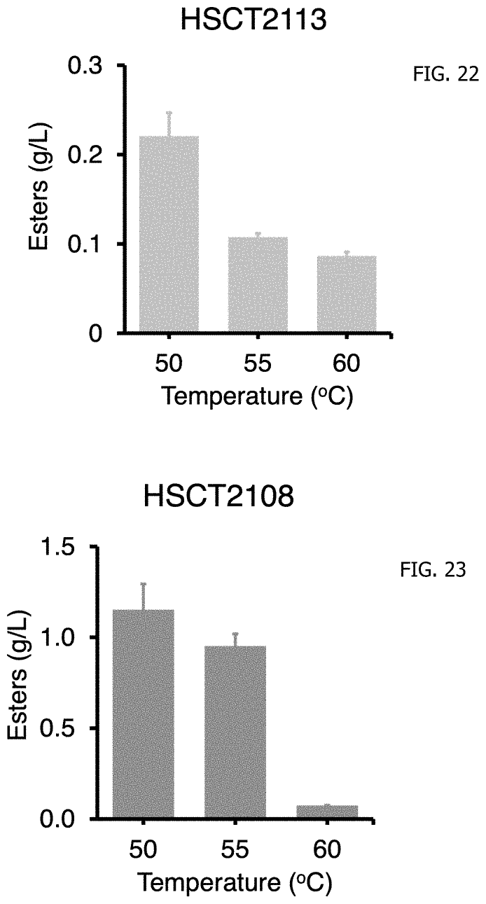

To further elucidate the effect of thermostability of CATs on ester production, we characterized the performance of HSCT2113 and HSCT2108 at various elevated temperatures compatible with C. thermocellum growth as shown in FIGS. 22 and 23. Interestingly, HSCT2113 increased the ester production up to 220 mg/L at 50° C., about 2-fold higher than 55° C. (FIG. 4B). In contrast, HSCT2108 produced esters at relatively similar level of about 1 g/L at 50 and 55° C., while the production was reduced to 74 mg/L at 60° C. (FIG. 4C). Proteolysis is a common cellular process to degrade and remove denatured or misfolded proteins. If the cell growth temperature affected integrity of the CATs, their intracellular abundances would be altered. To investigate changes in the intracellular abundance of CATs, we analyzed and compared proteomes across the two representative strains expressing CATec3, the wildtype (HSCT2107) versus the Y20F mutant (HSCT2108). Since the only difference between these two strains is one amino acid substitution, we could reliably quantify the relative abundance of each CAT by comparing tryptic peptide fragments. The result showed that CATec3 Y20F was 2.2-fold (average difference between peptide abundances) more abundant than CATec3. Because CATec3 Y20F has a 7° C. higher melting temperature than CATec3, the intracellular protein abundance change might imply a lesser degree of denaturation or misfolding due to higher protein thermostability. Taken altogether, CAT thermostability is critical for robust and efficient ester production in thermophiles by maintaining its intracellular protein abundance.

### Working Example 3: Catalytic Efficiency

Catalytic efficiency of CATec3 Y20F towards multiple alcohol substrates. The catalytic efficiency was measured from the kinetic reactions performed at 50° C. The co-substrate, acetyl-CoA, was supplemented at the saturated concentration of 2 mM. The values represent average±standard deviation from at least three biological replicates.

TABLE 1

| Alcohol | | | |
|---|---|---|---|
| substrates | k_{cat} (1/s) | K_{M} (mM) | k_{cat}/K_{M} (1/M/s) |
| Ethanol* | 0.8 ± 1.0 | 1232.5 ± 1986.3 | 0.6 ± 1.3 |
| Butanol | 1.9 ± 0.4 | 195.7 ± 87.3 | 10.5 ± 5.1 |
| Isobutanol | 2.3 ± 0.1 | 180.1 ± 21.7 | 13.0 ± 1.7 |
| Prenol | 2.7 ± 0.3 | 101.0 ± 21.6 | 26.4 ± 6.6 |
| Furfuryl alcohol | 1.1 ± 0.1 | 37.1 ± 9.8 | 29.1 ± 8.4 |
| Pentanol | 3.9 ± 1.0 | 64.7 ± 33.6 | 59.1 ± 19.3 |
| Isoamyl alcohol | 2.8 ± 0.3 | 59.9 ± 26.6 | 46.0 ± 11.9 |
| Benzyl alcohol | 1.6 ± 0.1 | 12.2 ± 1.6 | 130.3 ± 17.8 |
| Hexanol | 6.4 ± 0.4 | 29.2 ± 4.0 | 219.5 ± 33.5 |
| 3-cis-Hexen-1-ol | 2.3 ± 0.2 | 6.4 ± 2.2 | 360.1 ± 129.4 |
| Phenylethyl | 6.1 ± 0.1 | 10.6 ± 0.4 | 577.1 ± 26.2 |
| alcohol | | | |
| 3-Methoxybenzyl | 18.8 ± 0.9 | 10.0 ± 1.2 | 1,891.6 ± 248.8 |
| alcohol | | | |
| Geraniol | 8.0 ± 0.2 | 4.1 ± 0.4 | 1,975.7 ± 186.8 |
| Citronellol | 7.7 ± 0.2 | 3.2 ± 0.3 | 2,400.9 ± 247.9 |
| Nerol | 2.2 ± 0.04 | 4.1 ± 0.8 | 4,119.5 ± 753.0 |
| Chloramphenicol | 105.5 ± 6.9 | 0.2 ± 0.05 | 491,901.5 ± 115,286.2 |

*Calculation of the parameters against ethanol were not statistically practical due to the low affinity.

### Test Methods

To determine in vitro melting temperatures and catalytic efficiencies, His-tagged CATs were purified and characterized using know methods. In the 5,5′-dithiobis-2-nitrobenzoic acid (DTNB) assay, final enzyme concentrations of 0.05-0.1 μg/mL and 5-10 μg/mL were used for the reactions with chloramphenicol and alcohols, respectively. For heat inactivation experiments, 50 μL of the purified CATs were incubated at the temperatures from 50 to 70° C. in a thermocycler for an hour, with the lid temperature set at 70° C. Residual activity was measured at 37° C. using chloramphenicol and acetyl-CoA as substrates and normalized by the activity of the samples incubated at 50° C. To determine Michaelis-Menten kinetics, concentrations of the alcohol substrates varied as follows: (i) 0-400 mM for ethanol, butanol, and isobutanol, (ii) 0-100 mM for pentanol, isoamyl alcohol, 3-cis-hexen-1-ol, prenol, and furfuryl alcohol, (iii) 0-2 mM for octanol, (iv) 0-0.2 mM for decanol, (v) 0-50 mM for hexanol, citronellol, farnesol, and nerol, (vi) 0-40 mM for 3-methoxybenzyl alcohol, benzyl alcohol, and geraniol, and (vii) 0-20 mM 2-phenylethyl alcohol. For the alcohols with low solubility, 10% (w/v) DMSO was supplemented in the reaction solution. The enzyme reactions were held at 50° C. in a BioTek microplate reader for at least 30 minutes with measurements every one minute. The kinetic parameters were calculated using a non-linear regression method known to one of skill in the art.

Growth of E. coli: E. coli strains were grown in lysogeny broth (LB) medium or M9 hybrid medium containing glucose as a carbon source and 5 g/L yeast extract supplemented with 100 μg/mL ampicillin and/or 50 μg/mL kanamycin when appropriate.

E. coli ester production: For batch cultures, tube-scale alcohol conversions were performed in 4 mL M9 medium containing 10 g/L glucose with addition of 1 mL of hexadecane for in situ extraction at 37° C. 0.1 mM isopropyl β-D-1-thiogalactopyranoside (IPTG) was initially added to induce expression of CATec3 Y20F. Alcohols were supplemented in the initial medium, and the product yield and titer were measured at 12 h, 24 h, and 48 h time points. For fed-batch cultures designed to achieve high-level conversion of alcohols (i.e., isoamyl alcohol, phenylethyl alcohol), cells were grown micro-aerobically in a 125 mL screw capped shake flask with a working volume of 20 mL M9 medium containing 25 g/L glucose and 10 mL hexadecane. A volume of 25-50 μL of the alcohols (≥98% purity) were added to the culture at 6 h, 9 h, 12 h, 15 h, and 24 h time points with a working concentration of 2 g/L per addition.

Growth of C. thermocellum: C. thermocellum strains were cultured in an anaerobic chamber (Sheldon manufacturing, OR, USA) with an anaerobic gas mixture (90% N₂, 5% CO₂, 5% H₂) or rubber stopper sealed anaerobic Balch tubes outside the chamber. For C. thermocellum transformation, CTFuD or CTFuD-NY media was used. The CTFuD medium contained 2 g/L yeast extract while CTFuD-NY used vitamins and trace elements instead of the yeast extract. To maintain the plasmids in C. thermocellum, 10 μg/mL thiamphenicol was supplemented. For alcohol conversion experiments with C. thermocellum, strains were grown in a defined C-MTC medium as previously described. C. thermocellum cells were transformed by electroporation as previously described. A series of two consecutive exponential pulses were applied using the electroporation system (cat #45-0651, BTX Technologies Inc., MA, USA) set at 1.8 kV, 25 μF, and 350Ω, which usually resulted in a pulse duration of 7.0-8.0 ms.

C. thermocellum ester production: Tube-scale cellulose fermentation was performed in the batch mode as previously described (29). Briefly, 19 g/L of Avicel PH-101 was used as a sole carbon source in a 16 mL culture volume. 0.8 mL of overnight cell culture was inoculated in 15.2 mL of C-MTC medium, and 4 mL hexadecane was added in the anaerobic chamber. Each tube contained a small magnetic stirrer bar to homogenize cellulose, and the culture was incubated in a water bath connected with a temperature controller and a magnetic stirring system. Alcohols were fed to the culture at 36 h time point when cells entered early stationary growth phase. pH was adjusted to between 6.4 and 7.8 with 5 M KOH injection.

In vivo screening of CAT_{Sa} variants: To prepare pre-cultures, single colonies from LB agar plates were first inoculated into 100 μL of LB in 96-well microplates using sterile pipette tips. The pre-cultures were then grown overnight at 37° C. and 400 rpm in an incubating microplate shaker (Fisher Scientific, PA, USA). Next, 5% (v/v) of pre-cultures were inoculated into 100 μL of the M9 hybrid media containing 20 g/L of glucose, 0.1 mM of IPTG, and 2 g/L of isobutanol in a 96-well microplate with hexadecane overlay, containing isoamyl alcohol as an internal standard, in a 1:1 (v/v) ratio. The microplates were sealed with a plastic adhesive sealing film, SealPlate® (EXCEL Scientific, Inc., CA, USA) and incubated at 37° C. and 400 rpm for 24 h in an incubating microplate shaker. Samples from the hexadecane layer were collected and subjected to GC/MS for ester identification and quantification.

Quantification of Esters: Gas chromatography (HP 6890, Agilent, CA, USA) equipped with mass spectroscopy (HP 5973, Agilent, CA, USA) was used to quantify esters. A Zebron ZB-5 (Phenomenex, CA, USA) capillary column (30 m×0.25 mm×0.25 μm) was used with helium as the carrier gas at a flow rate of 0.5 mL/min. The oven temperature program was set as follows: 50° C. initial temperature, 1° C./min ramp up to 58° C., 25° C./min ramp up to 235° C., 50° C./min ramp up to 300° C., and 2-minutes bake-out at 300° C. 1 μL sample was injected into the column with the splitless mode at an injector temperature of 280° C. For the MS system, selected ion mode (SIM) was used to detect and quantify esters. As an internal standard, 10 mg/L n-decane were added in initial hexadecane layer and detected with m/z 85, 99, and 113 from 12 to 15 minute retention time range.

### Working Example 4: Demonstration of Engineered C. thermocellum Harboring the Engineered CAT For Direct Conversion of Cellulosic Biomass to Esters

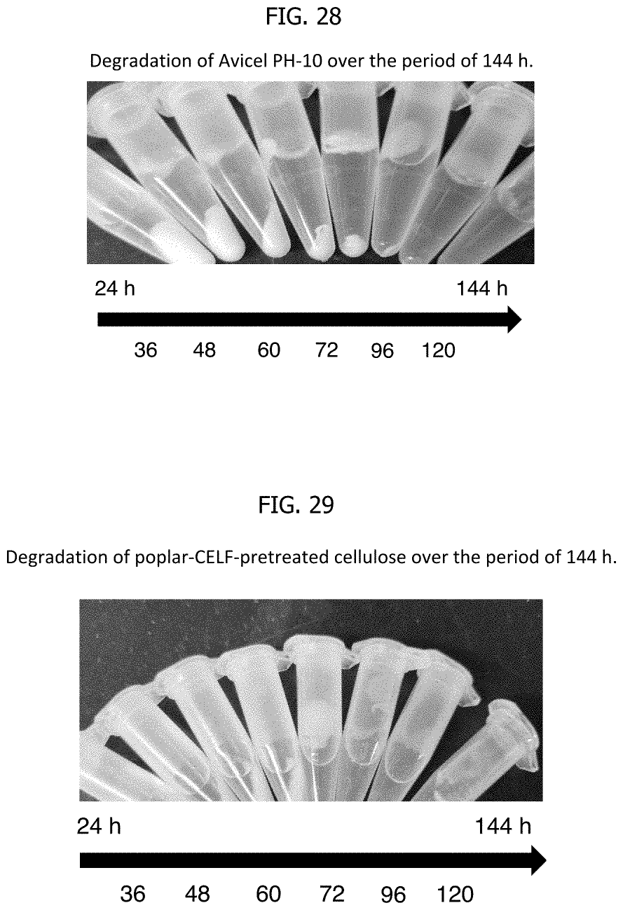

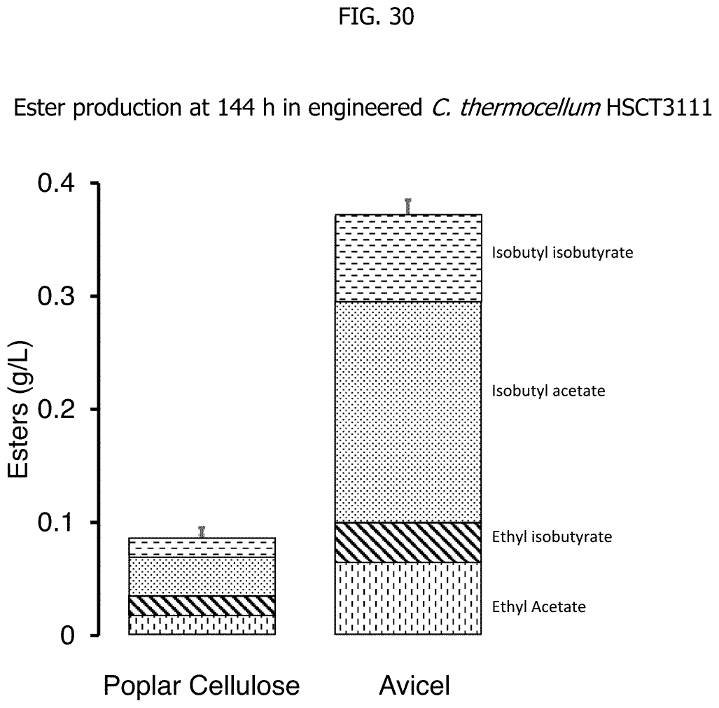

Two carbohydrate esterase genes (Clo1313_0613 and Clo1313_0693) and one lactate dehydrogenase gene (Clo1313_1160) were disrupted from the genome of C. thermocellum DSM1313 Δhpt to create the strain HSCT3009(1). This strain was engineered to eliminate ester degradation and production of the common byproduct lactate. The plasmid pHS0070 carrying the engineered gene CATec3 Y20F was then transformed into HSCT3009 by electroporation to create the strain HSCT3111. Next, the strain HSCT3111 was characterized in rubber stopper sealed anaerobic Balch tubes including 15.2 mL of defined C-MTC media containing 19 g/L cellulosic biomass (i.e., commercial Avicel PH-101 and poplar-CELF-pretreated biomass), 0.8 mL of overnight cell culture, and 4 mL hexadecane. Each tube contained a small magnetic stirrer bar to homogenize cellulose and the culture was incubated in a temperature controlled water bath connected at 55° C. with stirring. Following pH adjustment with 70 μL of 5 M KOH injection, 800 μL of cell culture and 200 μL of hexadecane layer were sampled every 12 hours for measuring cell growth and extracellular metabolites. Cell growth was determined by measuring pellet protein with the Bradford assay known in the art. Residual cellulose was determined by the phenol-sulfuric acid method. A high-performance liquid chromatography system was used to quantify extracellular metabolites such as sugars, organic acids, and alcohols in the cell culture. Gas chromatography coupled with mass spectroscopy was used to quantify esters in the hexadecane layer. FIGS. 28 and 29 show samples of cell cultures' degrading insoluble fraction of avicel and poplar-CELF-pretreated cellulose over a period of 144 h, respectively. The residual sugar amount was <0.1 g/L, corresponding about 19 g/L cellulose consumption. FIG. 30 shows the different types of esters produced from samples collected at 144 h, an ethyl acetate, an ethyl isobutyrate, isobutyl acetate, and isobutyl isobutyrate.

The modified CATs herein function as robust and efficient AATs that exhibit high compatibility to a broad range of pathways and microbial hosts. The modified CATs are capable of producing designer esters in both mesophilic and thermophilic microorganisms with high efficiency, robustness, and compatibility. Using proteomics and comparative analysis of the modified CATs, we found that the CAT robustness with enhanced thermostability provides superior efficient ester production in thermophiles by maintaining high level of intracellular CAT abundance. Designer bioesters can be produced by using the modified CATs and either co-feeding fermentable sugars or cellulose and alcohols or carboxylic acids as demonstrated here or via natural fermentative processes which produce alcohols natively. This microbial production of esters presents a renewable and sustainable route to synthesize such chemicals.

It should be noted that the embodiments are not limited in their application or use to the details of construction and arrangement of parts and steps illustrated in the drawings and description. Features of the illustrative embodiments and variants may be implemented or incorporated in other embodiments, variants, and modifications, and may be practiced or carried out in various ways. Furthermore, unless otherwise indicated, the terms and expressions employed herein have been chosen for the purpose of describing the illustrative embodiments of the present invention for the convenience of the reader and are not for the purpose of limiting the invention. Having described the invention in detail and by reference to preferred embodiments thereof, it will be apparent that modifications and variations are possible without departing from the scope of the invention which is defined in the appended claims.

TABLE 1

| SEQ | | | | |
|---|---|---|---|---|
| ID. | SEQ | Amino Acid | | |
| NO. | Name | Sequence | Type | Origin |
| SEQ | CAT_ | MNFNKIDLDNWKRKE | A | Staphylococcus |
| ID | Sa | IFNHYLNQQTTFSIT | | aureus |
| NO. | | TEIDISVLYRNIKQE | | |
| 1 | | GYKFYPAFIFLVTRV | | |
| | | INSNTAFRTGYNSDG | | |
| | | ELGYWDKLEPLYTIF | | |
| | | DGVSKTFSGIWTPVK | | |
| | | NDFKEFYDLYLSDVE | | |
| | | KYNGSGKLFPKTPIP | | |
| | | ENAFSLSIIPWTSFT | | |
| | | GFNLNINNNSNYLLP | | |
| | | IITAGKFINKGNSIY | | |
| | | LPLSLQVHHSVCDGY | | |
| | | HAGLFMNSIQELSDR | | |
| | | PNDWLL | | |
| SEQ | CAT_ | MNFHKVNWNEWERKE | A | Thermoactinomyces |
| ID | THEACI | TFHHFLNQQTTFSMT | | sp. |
| NO. | | TEIDITALYARIKQK | | |
| 2 | | GFKFYPAFLYWTRWN | | |
| | | SHTAFRMGYNHKREF | | |
| | | GCWDQLHPLYTIFDR | | |
| | | ESEMFSGIWTMAEGD | | |
| | | FKAFYRLYLTDVERY | | |
| | | GGSGKLFPKTPIPEN | | |
| | | AFSVSMIPWTSFTGF | | |
| | | NLNIHNQRDYLLPIV | | |
| | | TAGKWIRHGRSIRLP | | |
| | | VALQVHHAVCDGYHA | | |
| | | GMFMNAVQEWADHPE | | |
| | | EWL | | |
| SEQ | CAT_ | MRFNKIDINNWERKE | A | Lysinibacillus |
| ID | LYS | IFNHFLNQQTSFSIT | | boronitolerans |
| NO. | | RTIDITELYKITKDK | | |
| 3 | | GYKFYPVLIFLITHV | | |
| | | ANSHKHFRMNFNSAG | | |
| | | EFGYWDKWPMYTIFD | | |
| | | KQSELFSAIYTNTDE | | |
| | | GFKKFYENYISDTEK | | |
| | | FNGKGKLFPKTPIPE | | |
| | | NWNISMIPWTSFTGF | | |
| | | NLNVNNSPNYCLPII | | |
| | | TAGRFINKTSNIYLP | | |
| | | LSLQVHHSVCDGYHA | | |
| | | ALFMDRFQTLV | | |
| SEQ | CAT_ | MTFTVINRRTWKREE | A | Bacillus sp. |
| ID | BACI | VFSHYIKQKTSFSLT | | |
| NO. | | TELEVDVLYKRVKQK | | |
| 4 | | GYTFYPAFLYLVTSV | | |
| | | VNKHVAFRMSFNQEG | | |
| | | GLGYWSQLEPVYTIF | | |
| | | HEKTKLFSGIWTSMN | | |
| | | RDFNHFHTSYLQDVM | | |
| | | TYQGSKALFPKKHLP | | |
| | | ENTVSVSMIPWTSFT | | |
| | | GFNLMIQQDTNYLLP | | |
| | | IVTAGKLIEKNQTLY | | |
| | | LPVSLQVHHAVCDGY | | |
| | | HASMFMNDCQQLANQ | | |
| | | AHEWI | | |
| SEQ | CAT_ | MKFNSINRDNWDRKE | A | Staphylococcus |
| ID | BAOCE | YFEHYIQQQTTFSLT | | aureus |
| NO. | | NEINITTLMKNLKKK | | |
| 5 | | NYKLYPAFIFMVTKI | | |
| | | VNAHREFRINFNSEG | | |
| | | NLGYWTEICPLYTIF | | |
| | | DKKTHTFSGIWSPNL | | |
| | | SIFSEFHSQYEKDAE | | |
| | | EYNGTGRLFPKIPIP | | |
| | | DNTIPISMIPWSSFT | | |
| | | AFNLNINNGGDFLLP | | |
| | | IITGGKYSQVNDEFF | | |
| | | LPVSLQMHHAVCDGY | | |
| | | HAGVFMNDLQRLADE | | |
| | | SADWI | | |
| SEQ | CAT_ | MTFNPITLENWERKE | A | Geomicrobium sp. |
| ID | GEO | YFNHYLNQQTTFSMT | | |
| NO. | | TDIEISSFKAAIKRK | | |
| 6 | | GYKFYPTFIYMVTEV | | |
| | | INANESFRISFNEEG | | |
| | | KLGYWEKLIPLYTVF | | |
| | | DDQKQSFSNLWTDST | | |
| | | GNVLTFQEDYDRDVA | | |
| | | EYNHIGGLFPKTPIP | | |
| | | ANTFPISMIPWNSFS | | |
| | | GFNLNVGNGGNFLLP | | |
| | | IITAGKYYSKGTATY | | |
| | | LPVSLQVHHAVCDGY | | |
| | | HAASFMNQLQELANT | | |
| | | TNEWL | | |
| SEQ | CAT5_ | MTFNIINLETWDRKE | A | Staphylococcus |
| ID | STAAU | YFNHYFNQQTTYSVT | | aureus |
| NO. | | KELDITLLKSMIKNK | | |
| 7 | | GYELYPALIHAIVSV | | |
| | | INRNKVFRTGINSEG | | |
| | | NLGYWDKLEPLYTVF | | |
| | | NKETENFSNIWTESN | | |
| | | ASFTLFYNSYKNDLI | | |
| | | KYKDKNEMFPKKPIP | | |
| | | ENTVPISMIPWIDFS | | |
| | | SFNLNIGNNSRFLLP | | |
| | | IITIGKFYSKDDKIY | | |
| | | LPFPLQVHHAVCDGY | | |
| | | HVSLFMNEFQNIIR | | |
| SEQ | CAT_ | MTFNIIELENWDRKE | A | Streptococcus |
| ID | STRAG | YFEHYFNQQTTYSIT | | agalactiae |
| NO. | | KEIDITLFKDMIKKK | | |
| 8 | | GYEIYPSLIYAIMEW | | |
| | | NKNKVFRTGINSENK | | |
| | | LGYWDKLNPLYTVFN | | |
| | | KQTEKFTNIWTESDK | | |
| | | NFISFYNNYKNDLLE | | |
| | | YKDKEEMFPKKPIPE | | |
| | | NTIPISMIPWIDFSS | | |
| | | FNLNIGNNSSFLLPI | | |
| | | ITIGKFYSENNKIYI | | |
| | | PVALQLHHSVCDGYH | | |
| | | ASLFMNEFQDIIHRV | | |
| | | DDWI | | |
| SEQ | CAT4_ | MTFNIIKLENWDRKE | A | Staphylococcus |
| ID | STAAU | YFEHYFNQQTTYSIT | | aureus |
| NO. | | KEIDITLFKDMSKKK | | |
| 9 | | GYEIYPSLIYAIMEV | | |
| | | VNKNKVFRTGINSEN | | |
| | | KLGYWDKLNPLYTVF | | |
| | | NKQTEKFTNIWTESD | | |
| | | NNFTSFYNNYKNDLL | | |
| | | EYKDKEEMFPKKPIP | | |
| | | ENTLPISMIPWIDFS | | |
| | | SFNLNIGNNSNFLLP | | |
| | | MTIGKFYSENNKIYI | | |
| | | PVALQLHHAVCDGYH | | |
| | | ASLFINEFQDIIKKV | | |
| | | DDWI | | |
| SEQ | CAT3_ | MTFNIIKLENWDRKE | A | Staphylococcus |
| ID | STAAU | YFEHYFNQQTTYSIT | | aureus |
| NO. | | KEIDITLFKDMIKKK | | |
| 10 | | GYEIYPSLIYAIMEW | | |
| | | NKNKVFRTGINSENK | | |
| | | LGYWDKLNPLYTVFN | | |
| | | KQTEKFTNIWTESDN | | |
| | | NFTSFYNNYKNDLFE | | |
| | | YKDKEEMFPKKPIPE | | |
| | | NTIPISMIPWIDFSS | | |
| | | FNLNIGNNSSFLLPI | | |
| | | ITIGKFYSENNKIYI | | |
| | | PVALQLHHAVCDGYH | | |
| | | ASLFINEFQDIINKV | | |
| | | DDWI | | |
| SEQ | CAT_ | MTFNIIKLENWDRKE | A | Staphylococcus |
| ID | STAIN | YFEHYFNQQTTYSIT | | intermedius |
| NO. | | KEIDITLFKDMIKKK | | |
| 11 | | GYEIYPSLIYAIMEW | | |
| | | NKNKVFRTGINSENK | | |
| | | LGYWDKLNPLYTVFN | | |
| | | KQTEKFTNIWTESDN | | |
| | | NFTSFYNNYKNDLFE | | |
| | | YKDKEEMFPKKPIPE | | |
| | | NTIPISMIPWIDFSS | | |
| | | FNLNIGNNSSFLLPI | | |
| | | ITIGKFYSENNKIYI | | |
| | | PVALQLHHAVCDGYH | | |
| | | ASLFINEFQDIIKKV | | |
| | | DDWI | | |
| SEQ | CAT_ | MFKQIDENYLRKEHF | A | Bacillus pumilus |
| ID | BACPU | HHYMTLTRCSYSLVI | | |
| NO. | | NLDITKLHAILKEKK | | |
| 12 | | LKVYPVQIYLLARAV | | |
| | | QKIPEFRMDQVNDEL | | |
| | | GYWEILHPSYTILNK | | |
| | | ETKTFSSIWTPFDEN | | |
| | | FAQFYKSCVADIETF | | |
| | | SKSSNLFPKPHMPEN | | |
| | | MFNISSLPWIDFTSF | | |
| | | NLNVSTDEAYLLPIF | | |
| | | TIGKFKVEEGKIILP | | |
| | | VAIQVHHAVCDGYHA | | |
| | | GQYVEYLRWLIEHCD | | |
| | | EWLNDSLHIT | | |
| SEQ | CAT_ | MNFNLIDINHWSRKP | A | Clostridium |
| ID | CLOBU | YFEHYLNNVKCTYSM | | butyricum |
| NO. | | TANIEITDLLYEIKL | | |
| 13 | | KNIKFYPTLIYMIAT | | |
| | | WNNHKEFRICFDHKG | | |
| | | SLGYWDSMNPSYTIF | | |
| | | HKENETFSSIWTEYN | | |
| | | KSFLRFYSDYLDDIK | | |
| | | NYGNIMKFTPKSNEP | | |
| | | DNTFSVSSIPWVSFT | | |
| | | GFNLNVYNEGTYLIP | | |
| | | IFTAGKYFKQENKIF | | |
| | | IPISIQVHHAICDGY | | |
| | | HASRFINEMQELAFS | | |
| | | FQEWLENK | | |
| SEQ | CAT1_ | MKFNLIDIEDWNRKP | A | Clostridium |
| ID | CL0PF | YFEHYLNAVRCTYSM | | perfringens |
| NO. | | TANIEITGLLREIKL | | |
| 14 | | KGLKLYPTLIYIITT | | |
| | | VVNRHKEFRTCFDQK | | |
| | | GKLGYWDSMNPSYTV | | |
| | | FHKDNETFSSIWTEY | | |
| | | DENFPRFYYNYLEDI | | |
| | | RNYSDVLNFMPKTGE | | |
| | | PANTINVSSIPWVNF | | |
| | | TGFNLNIYNDATYLI | | |
| | | PIFTLGKYFQQDNKI | | |
| | | LLPMSVQVHHAVCDG | | |
| | | YHISRFFNEAQELAS | | |
| | | NYETWLGEK | | |
| SEQ | CAT_ | MVFEKIDKNSWNRKE | A | Clostridioides |
| ID | CLODI | YFDHYFASVPCTYSM | | difficile |
| NO. | | TVKVDITQIKEKGMK | | |
| 15 | | LYPAMLYYIAMIVNR | | |
| | | HSEFRTAINQDGELG | | |
| | | IYDEMIPSYTIFHND | | |
| | | TETFSSLWTECKSDF | | |
| | | KSFLADYESDTQRYG | | |
| | | NNHRMEGKPNAPENI | | |
| | | FNVSMIPWSTFDGFN | | |
| | | LNLQKGYDYLIPIFT | | |
| | | MGKIIKKDNKIILPL | | |
| | | AIQVHHAVCDGFHIC | | |
| | | RFVNELQELIIVTQV | | |
| | | CL | | |
| SEQ | CAT_ | MQFTKIDINNWTRKE | A | Campylobacter coli |
| ID | CAMCO | YFDHYFGNTPCTYSM | | |
| NO. | | TVKLDISKLKKDGKK | | |
| 16 | | LYPTLLYGVTTIINR | | |
| | | HEEFRTALDENGQVG | | |
| | | VFSEMLPCYTVFHKE | | |
| | | TETFSSIWTEFTADY | | |
| | | TEFLQNYQKDIDAFG | | |
| | | ERMGMSAKPNPPENT | | |
| | | FPVSMIPWTSFEGFN | | |
| | | LNLKKGYDYLLPIFT | | |
| | | FGKYYEEGGKYYIPL | | |
| | | SIQVHHAVCDGFHVC | | |
| | | RFLDELQDLLNK | | |
| SEQ | CAT_ | MEFRLVDLKTWKRKE | A | Vibrio anguillarum |
| ID | VIBAN | YFTHYFESVPCTYSM | | |
| NO. | | TVKLDITTIKTGKAK | | |
| 17 | | LYPALLYAVSTVVNR | | |
| | | HEEFRMTVDDEGQIG | | |
| | | IFSEMMPCYTIFQKD | | |
| | | TEMFSNIWTEYIGDY | | |
| | | TEFCKQYEKDMQQYG | | |
| | | ENKGMMAKPNPPVNT | | |
| | | FPVSMIPWTTFEGFN | | |
| | | LNLQKGYGYLLPIFT | | |
| | | FGRYYEENGKYWIPL | | |
| | | SIQVHHAVCDGFHTC | | |
| | | RFINELQDVIQSLQN | | |
| | | HGGDEE | | |
| SEQ | CAT_ | MDAPIPTPAPIDLDT | A | Streptomyces |
| ID | STRAC | WPRRQHFDHYRRRVP | | acrimycini |
| NO. | | CTYAMTVEVDVTAFA | | |
| 18 | | AALRRSPRKSYLAQV | | |
| | | WALATVVNRHEEFRM | | |
| | | CLNSSGDPAVWPVVH | | |
| | | PAFTVFNPERETFAC | | |
| | | LWAPYDPDFGTFHDT | | |
| | | AAPLLAEHSRATDFF | | |
| | | PQGNPPPNAFDVSSL | | |
| | | PWVSFTGFTLDIRDG | | |
| | | WDHLAPIFTLGRYTE | | |
| | | RDTRLLLPLSVQIHH | | |
| | | AAADGFHTARLTNEL | | |
| | | QTLLADPAWL | | |
| SEQ | CAT2_ | MNFTRIDLNTWNRRE | A | Escherichia coli |
| ID | ECOLX | HFALYRQQIKCGFSL | | |
| NO. | | TTKLDITALRTALAE | | |
| 19 | | TGYKFYPLMIYLISR | | |
| | | AVNQFPEFRMALKDN | | |
| | | ELIYWDQSDPVFTVF | | |
| | | HKETETFSALSCRYF | | |
| | | PDLSEFMAGYNAVTA | | |
| | | EYQHDTRLFPQGNLP | | |
| | | ENHLNISSLPWVSFD | | |
| | | GFNLNITGNDDYFAP | | |
| | | VFTMAKFQQEGDRVL | | |
| | | LPVSVQVHHAVCDGF | | |
| | | HAARFINTLQLMCDN | | |
| | | ILK | | |
| SEQ | CAT2_ | MNFTRIDLNTWNRRE | A | Haemophilus |
| ID | HAEIF | HFALYRQQIKCGFSL | | influenzae |
| NO. | | TTKLDITAFRTALAE | | |
| 20 | | TDYKFYPVMIYLISR | | |
| | | VVNQFPEFRMAMKDN | | |
| | | ALIYWDQTDPVFTVF | | |
| | | HKETETFSALFCRYC | | |
| | | PDISEFMAGYNAVMA | | |
| | | EYQHNTALFPQGALP | | |
| | | ENHLNISSLPWVSFD | | |
| | | GFNLNITGNDDYFAP | | |
| | | VFTMAKFQQEDNRVL | | |
| | | LPVSVQVHHAVCDGF | | |
| | | HAARFINTLQMMCDN | | |
| | | ILK | | |
| SEQ | CAT3_ | MNYTKFDVKNWVRRE | A | Escherichia coli |
| ID | ECOLX | HFEFYRHRLPCGFSL | | |
| NO. | | TSKIDITTLKKSLDD | | |
| 21 | | SAYKFYPVMIYLIAQ | | |
| | | AVNQFDELRMAIKDD | | |
| | | ELIVWDSVDPQFTVF | | |
| | | HQETETFSALSCPYS | | |
| | | SDIDQFMVNYLSVME | | |
| | | RYKSDTKLFPQGVTP | | |
| | | ENHLNISALPWVNFD | | |
| | | SFNLNVANFTDYFAP | | |
| | | IITMAKYQQEGDRLL | | |
| | | LPLSVQVHHAVCDGF | | |
| | | HVARFINRLQELCNS | | |
| | | KLK | | |
| SEQ | CAT_ | MDTKRVGILVVDLSQ | A | Proteus mirabilis |
| ID | PROMI | WGRKEHFEAFQSFAQ | | |
| NO. | | CTFSQTVQLDITSLL | | |
| 22 | | KTVKQNGYKFYPTFI | | |
| | | YIISLLVNKHAEFRM | | |
| | | AMKDGELVIWDSVNP | | |
| | | GYNIFHEQTETFSSL | | |
| | | WSYYHKDINRFLKTY | | |
| | | SEDIAQYGDDLAYFP | | |
| | | KEFIENMFFVSANPW | | |
| | | VSFTSFNLNMANINN | | |
| | | FFAPVFTIGKYYTQG | | |
| | | DKVLMPLAIQVHHAV | | |
| | | CDGFHVGRLLNEIQQ | | |
| | | YCDEGCK | | |
| SEQ | CAT1_ | MEKKITGYTTVDISQ | A | Escherichia coli |
| ID | ECOLX | WHRKEHFEAFQSVAQ | | |
| NO. | | CTYNQTVQLDITAFL | | |
| 23 | | KTVKKNKHKFYPAFI | | |
| | | HILARLMNAHPEFRM | | |
| | | AMKDGELVIWDSVHP | | |
| | | CYTVFHEQTETFSSL | | |
| | | WSEYHDDFRQFLHIY | | |
| | | SQDVACYGENLAYFP | | |
| | | KGFIENMFFVSANPW | | |
| | | VSFTSFDLNVANMDN | | |
| | | FFAPVFTMGKYYTQG | | |
| | | DKVLMPLAIQVHHAV | | |
| | | CDGFHVGRMLNELQQ | | |
| | | YCDEWQGGA | | |
| SEQ | CAT_ | MEKKITGYTTVDISQ | A | Klebsiella sp. |
| ID | KLESP | WHRKEHFEAFQSVAQ | | |
| NO. | | CTYNQTVQLDITAFL | | |
| 24 | | KTVKKNKHKFYPAFI | | |
| | | HILARLMNAHPEFRM | | |
| | | AMKDGELVIWDSVHP | | |
| | | CYTVFHEQTETFSSL | | |
| | | WSEYHDDFRQFLHIY | | |
| | | SQDVACYGENLAYFP | | |
| | | KGFIENMFFVSANPW | | |
| | | VSFTSFDLNVAAMDN | | |
| | | FFAPVFTMGKYYTQG | | |
| | | DKVLMPLAIQVHHAV | | |
| | | CDGFHVGRMLNELQQ | | |
| | | YCDEWQGGA | | |
| SEQ | CAT4_ | MGNYFESPFRGKLLS | B | Pseudomonas |
| ID | PSEAE | EQVSNPNIRVGRYSY | | aeruginosa |
| NO. | | YSGYYHGHSFDDCAR | | |
| 25 | | YLMPDRDDVDKLVIG | | |
| | | SFCSIGSGAAFIMAG | | |
| | | NQGHRAEWASTFPFH | | |
| | | FMHEEPVFAGAVNGY | | |
| | | QPAGDTLIGHDVWIG | | |
| | | TEAMFMPGVRVGHGA | | |
| | | IIGSRALVTGDVEPY | | |
| | | AIVGGNPARTIRKRF | | |
| | | SDGDIQNLLEMAWWD | | |
| | | WPLADIEAAMPLLCT | | |
| | | GDIPALYRHWKQRQA | | |
| | | TA | | |
| SEQ | CAT4_ | MTNYFESPFKGKLLT | B | Escherichia coli |
| ID | ECOLX | EQVKNPNIKVGRYSY | | |
| NO. | | YSGYYHGHSFDDCAR | | |
| 26 | | YLLPDRDDVDQLIIG | | |
| | | SFCSIGSGAAFIMAG | | |
| | | NQGHRYDWVSSFPFF | | |
| | | YMNEEPAFAKSVDAF | | |
| | | QRAGDTVIGSDVWIG | | |
| | | SEAMIMPGIKIGHGA | | |
| | | VIGSRALVAKDVEPY | | |
| | | TIVGGNPAKSIRKRF | | |
| | | SEEEISMLLDMAWWD | | |
| | | WPLEQIKEAMPFLCS | | |
| | | SGIASLYRRWQGTSA | | |
| SEQ | CAT4_ | MTNYFDSPFKGKLLS | B | Klebsiella |
| ID | KLEAE | EQVKNPNIKVGRYSY | | aerogenes |
| NO. | | YSGYYHGHSFDDCAR | | |
| 27 | | YLFPDRDDVDKLIIG | | |
| | | SFCSIGSGASFIMAG | | |
| | | NQGHRYDWASSFPFF | | |
| | | YMQEEPAFSSALDAF | | |
| | | QKAGNTVIGNDVWIG | | |
| | | SEAMVMPGIKIGHGA | | |
| | | VIGSRSLVTKDVEPY | | |
| | | AIVGGNPAKKIKKRF | | |
| | | TDEEISLLLEMEWWN | | |
| | | WSLEKIKAAMPMLCS | | |
| | | SNIVGLHKYWLEFAV | | |
| SEQ | CAT4_ | MKNYFDSPFKGELLS | B | Morganella |
| ID | MORMO | EQVKNPNIKVGRYSY | | morganii |
| NO. | | YSGYYHGHSFDECAR | | |
| 28 | | YLHPDRDDVDKLIIG | | |
| | | SFCSIGSGASFIMAG | | |
| | | NQGHRHDWASSFPFF | | |
| | | YMQEEPAFSSALDAF | | |
| | | QRAGDTAIGNDVWIG | | |
| | | SEAMIMPGIKIGDGA | | |
| | | VIGSRSLVTKDVVPY | | |
| | | AIIGGSPAKQIKKRF | | |
| | | SDEEISLLMEMEWWN | | |
| | | WPLDKIKTAMPLLCS | | |
| | | SNIFGLHKYWREFVV | | |

What is claimed is:

1. A modified chloramphenicol acetyltransferase comprising a tyrosine residue 20 having a phenylalanine (Y20F) mutation.

2. The modified chloramphenicol acetyltransferase of claim 1, comprising additional amino acid mutation of a phenylalanine residue 97 having a tryptophan (F97W) and/or an alanine residue 138 having a threonine (A138T).

3. The modified chloramphenicol acetyltransferase of claim 2, wherein the chloramphenicol acetyltransferase is from one or more of the following: Staphylococcus aureus, Escherichia coli, Haemophilus influenzae, Clostridium butyricum, and Lysinibacillus boronitolerans.

4. The modified chloramphenicol acetyltransferase of claim 2, wherein the chloramphenicol acetyltransferase has a wild type melting temperature that is greater than 60° C. and a specific activity toward isobutanol at 50° C. of greater than 5 μmol/min/mg protein.

5. The modified chloramphenicol acetyltransferase of claim 1, wherein a post-mutation melting point is greater than 83° C.

6. The modified chloramphenicol acetyltransferase of claim 5, wherein a post-mutation specific activity toward isobutanol at 50° C. (k_{cat}/K_{M}) is greater than 5 1/M/s.

7. The modified chloramphenicol acetyltransferase of claim 6, wherein a post-mutation specific activity at 50° C. (k_{cat}/K_{M}) is greater than 5 1/M/s toward at least two different alcohols selected from the group consisting of butanol, prenol, furfuryl alcohol, pentanol, isoamyl alcohol, benzyl alcohol, hexanol, 3-cis-hexen-1-ol, phenylethyl alcohol, 3-methyoxybenzyl alcohol, geraniol, citronellol, and nerol.

8. The modified chloramphenicol acetyltransferase of claim 2, being CATsa Y20F, CATsa Y20F F97W, CATsa Y20F A138T, CATsa Y20F F97W A138T, CATec3 Y20F, or CATec3 F97W Y20F.

9. A microorganism harboring a modified chloramphenicol acetyltransferase comprising a tyrosine residue 20 having a phenylalanine (Y20F) mutation.

10. The microorganism of claim 9, wherein the microorganism is selected from the group consisting of Clostridium acetobutylicum, Clostridium propionicum, Clostridium kluyveri, Clostridium thermocellum, Clostridium clariflavum, Clostridium celluloyticum, Clostridium beijerinckii, Clostridium tyrobutyricum, Caldicellulosiruptor bescii, Thermoanaerobacterium thermosaccharolyticum, Lactococus lactis, Bacillus subtilis, Corynebacterium glutamicum, Acidothermus cellulolyticus, Pseudomonas putida, Escherichia coli, Ralstonia eutropha, Cyanobacteria spirulina, Acinetobacter baylyi, Aspergillus niger, Aspergillus pseudoterreus, Bacillus coagulans, Clostridium autoethanogenum, Clostridium carboxidivorans, Clostridium ljungdahlii, Cupriavidus necator, Pichia kudriavzevii, Pichia pastoris, Rhodosporidium toruloides, Saccharomyces cerevisiae, Yarrowia lipolytica, Zymomonas mobilis, Geobacillus caldoxylosilyticus, Geobacillus galactosidasius, Geobacillus icigianus, Geobacillus jurassicus, Geobacillus kaustophilus, Geobacillus lituanicus, Geobacillus stearothermophilus, Geobacillus subterraneus, Geobacillus thermantarcticus, Geobacillus thermocatenulatus, Geobacillus thermodenitrificans, Geobacillus thermoglucosidasius, Geobacillus G. thermoleovorans, Geobacillus toebii, Geobacillus uzenensis, Geobacillus vulcani, Geobacillus LC300, and combinations thereof.

11. A method of producing esters comprising:
providing a microorganism harboring a modified chloramphenicol acetyltransferase comprising a tyrosine residue 20 having a phenylalanine (Y20F) mutation in an environment suitable for the microorganism to produce an ester; and
feeding the microorganism a sugar or a cellulose; and
feeding the microorganism an alcohol and/or a carboxylic acid.

12. The method of claim 11, comprising extracting the ester to maintain non-toxic ester levels in the system.

13. The method of claim 11, wherein feeding comprises feeding the microorganism a mixture of alcohols to produce a plurality of esters.

14. The method of claim 13, wherein the mixture of alcohols has a preselected concentration for each alcohol to produce a preselected ester profile.

15. The method of claim 11, wherein the microorganism is a mesophilic microorganism.

16. The method of claim 11, wherein the microorganism is a thermophilic microorganism.

17. The method of claim 11, wherein the microorganism is selected from the group consisting of Clostridium acetobutylicum, Clostridium propionicum, Clostridium kluyveri, Clostridium thermocellum, Clostridium clariflavum, Clostridium celluloyticum, Clostridium beijerinckii, Clostridium tyrobutyricum, Caldicellulosiruptor bescii, Thermoanaerobacterium thermosaccharolyticum, Lactococus lactis, Bacillus subtilis, Corynebacterium glutamicum, Acidothermus cellulolyticus, Pseudomonas putida, Escherichia coli, Ralstonia eutropha, Cyanobacteria spirulina, Acinetobacter baylyi, Aspergillus niger, Aspergillus pseudoterreus, Bacillus coagulans, Clostridium autoethanogenum, Clostridium carboxidivorans, Clostridium ljungdahlii, Cupriavidus necator, Pichia kudriavzevii, Pichia pastoris, Rhodosporidium toruloides, Saccharomyces cerevisiae, Yarrowia lipolytica, Zymomonas mobilis, Geobacillus caldoxylosilyticus, Geobacillus galactosidasius, Geobacillus icigianus, Geobacillus jurassicus, Geobacillus kaustophilus, Geobacillus lituanicus, Geobacillus stearothermophilus, Geobacillus subterraneus, Geobacillus thermantarcticus, Geobacillus thermocatenulatus, Geobacillus thermodenitrificans, Geobacillus thermoglucosidasius, Geobacillus G. thermoleovorans, Geobacillus toebii, Geobacillus uzenensis, Geobacillus vulcani, Geobacillus LC300, and combinations thereof.

18. The method of claim 11, comprising feeding the microorganism a carboxylic acid and/or an alcohol to produce a carboxylic acid ester.

19. The method of claim 11, wherein feeding occurs in a fed-batch system.

20. The method of claim 19, wherein the fed-batch system includes intermittent feeding of the alcohol of greater than 10 g/L.


SEQUENCE LISTING (28 sequences)

SEQ ID NO: 1 - protein, 216 aa, Staphylococcus aureus
  SITE (1)..(216): CAT_Sa, Type A
MNFNKIDLDNWKRKEIFNHYLNQQTTFSITTEIDISVLYRNIKQEGYKFYPAFIFLVTRVINSNTAFRTGYNSDGELGYWDKLEPLYTIFDGVSKTFSGIWTPVKNDFKEFYDLYLSDVEKYNGSGKLFPKTPIPENAFSLSIIPWTSFTGFNLNINNNSNYLLPIITAGKFINKGNSIYLPLSLQVHHSVCDGYHAGLFMNSIQELSDRPNDWLL

SEQ ID NO: 2 - protein, 215 aa, Thermoactinomyces sp.
  SITE (1)..(215): CAT_THEACI, Type A
MNFHKVNWNEWERKETFHHFLNQQTTFSMTTEIDITALYARIKQKGFKFYPAFLYVVTRVVNSHTAFRMGYNHKREFGCWDQLHPLYTIFDRESEMFSGIWTMAEGDFKAFYRLYLTDVERYGGSGKLFPKTPIPENAFSVSMIPWTSFTGFNLNIHNQRDYLLPIVTAGKWIRHGRSIRLPVALQVHHAVCDGYHAGMFMNAVQEWADHPEEWL

SEQ ID NO: 3 - protein, 208 aa, Lysinibacillus boronitolerans
  SITE (1)..(208): CAT_LYS, Type A
MRFNKIDINNWERKEIFNHFLNQQTSFSITRTIDITELYKITKDKGYKFYPVLIFLITHVANSHKHFRMNFNSAGEFGYWDKVVPMYTIFDKQSELFSAIYTNTDEGFKKFYENYISDTEKFNGKGKLFPKTPIPENVVNISMIPWTSFTGFNLNVNNSPNYCLPIITAGRFINKTSNIYLPLSLQVHHSVCDGYHAALFMDRFQTLV

SEQ ID NO: 4 - protein, 215 aa, Bacillus sp.
  SITE (1)..(215): CAT_BACI, Type A
MTFTVINRRTWKREEVFSHYIKQKTSFSLTTELEVDVLYKRVKQKGYTFYPAFLYLVTSVVNKHVAFRMSFNQEGGLGYWSQLEPVYTIFHEKTKLFSGIWTSMNRDFNHFHTSYLQDVMTYQGSKALFPKKHLPENTVSVSMIPWTSFTGFNLMIQQDTNYLLPIVTAGKLIEKNQTLYLPVSLQVHHAVCDGYHASMFMNDCQQLANQAHEWI

SEQ ID NO: 5 - protein, 215 aa, Staphylococcus aureus
  SITE (1)..(215): CAT_BAOCE, Type A
MKFNSINRDNWDRKEYFEHYIQQQTTFSLTNEINITTLMKNLKKKNYKLYPAFIFMVTKIVNAHREFRINFNSEGNLGYWTEICPLYTIFDKKTHTFSGIWSPNLSIFSEFHSQYEKDAEEYNGTGRLFPKIPIPDNTIPISMIPWSSFTAFNLNINNGGDFLLPIITGGKYSQVNDEFFLPVSLQMHHAVCDGYHAGVFMNDLQRLADESADWI

SEQ ID NO: 6 - protein, 215 aa, Geomicrobium sp.
  SITE (1)..(215): CAT_GEO, Type A
MTFNPITLENWERKEYFNHYLNQQTTFSMTTDIEISSFKAAIKRKGYKFYPTFIYMVTEVINANESFRISFNEEGKLGYWEKLIPLYTVFDDQKQSFSNLWTDSTGNVLTFQEDYDRDVAEYNHIGGLFPKTPIPANTFPISMIPWNSFSGFNLNVGNGGNFLLPIITAGKYYSKGTATYLPVSLQVHHAVCDGYHAASFMNQLQELANTTNEWL

SEQ ID NO: 7 - protein, 209 aa, Staphylococcus aureus
  SITE (1)..(209): CAT5_STAAU, Type A
MTFNIINLETWDRKEYFNHYFNQQTTYSVTKELDITLLKSMIKNKGYELYPALIHAIVSVINRNKVFRTGINSEGNLGYWDKLEPLYTVFNKETENFSNIWTESNASFTLFYNSYKNDLIKYKDKNEMFPKKPIPENTVPISMIPWIDFSSFNLNIGNNSRFLLPIITIGKFYSKDDKIYLPFPLQVHHAVCDGYHVSLFMNEFQNIIR

SEQ ID NO: 8 - protein, 215 aa, Streptococcus agalactiae
  SITE (1)..(215): CAT_STRAG, Type A
MTFNIIELENWDRKEYFEHYFNQQTTYSITKEIDITLFKDMIKKKGYEIYPSLIYAIMEVVNKNKVFRTGINSENKLGYWDKLNPLYTVFNKQTEKFTNIWTESDKNFISFYNNYKNDLLEYKDKEEMFPKKPIPENTIPISMIPWIDFSSFNLNIGNNSSFLLPIITIGKFYSENNKIYIPVALQLHHSVCDGYHASLFMNEFQDIIHRVDDWI

SEQ ID NO: 9 - protein, 215 aa, Staphylococcus aureus
  SITE (1)..(215): CAT4_STAAU, Type A
MTFNIIKLENWDRKEYFEHYFNQQTTYSITKEIDITLFKDMSKKKGYEIYPSLIYAIMEVVNKNKVFRTGINSENKLGYWDKLNPLYTVFNKQTEKFTNIWTESDNNFTSFYNNYKNDLLEYKDKEEMFPKKPIPENTLPISMIPWIDFSSFNLNIGNNSNFLLPIITIGKFYSENNKIYIPVALQLHHAVCDGYHASLFINEFQDIIKKVDDWI

SEQ ID NO: 10 - protein, 215 aa, Staphylococcus aureus
  SITE (1)..(215): CAT3_STAAU, Type A
MTFNIIKLENWDRKEYFEHYFNQQTTYSITKEIDITLFKDMIKKKGYEIYPSLIYAIMEVVNKNKVFRTGINSENKLGYWDKLNPLYTVFNKQTEKFTNIWTESDNNFTSFYNNYKNDLFEYKDKEEMFPKKPIPENTIPISMIPWIDFSSFNLNIGNNSSFLLPIITIGKFYSENNKIYIPVALQLHHAVCDGYHASLFINEFQDIINKVDDWI

SEQ ID NO: 11 - protein, 215 aa, Staphylococcus intermedius
  SITE (1)..(215): CAT_STAIN, Type A
MTFNIIKLENWDRKEYFEHYFNQQTTYSITKEIDITLFKDMIKKKGYEIYPSLIYAIMEVVNKNKVFRTGINSENKLGYWDKLNPLYTVFNKQTEKFTNIWTESDNNFTSFYNNYKNDLFEYKDKEEMFPKKPIPENTIPISMIPWIDFSSFNLNIGNNSSFLLPIITIGKFYSENNKIYIPVALQLHHAVCDGYHASLFINEFQDIIKKVDDWI

SEQ ID NO: 12 - protein, 220 aa, Bacillus pumilus
  SITE (1)..(220): CAT_BACPU, Type A
MFKQIDENYLRKEHFHHYMTLTRCSYSLVINLDITKLHAILKEKKLKVYPVQIYLLARAVQKIPEFRMDQVNDELGYWEILHPSYTILNKETKTFSSIWTPFDENFAQFYKSCVADIETFSKSSNLFPKPHMPENMFNISSLPWIDFTSFNLNVSTDEAYLLPIFTIGKFKVEEGKIILPVAIQVHHAVCDGYHAGQYVEYLRWLIEHCDEWLNDSLHIT

SEQ ID NO: 13 - protein, 219 aa, Clostridium butyricum
  SITE (1)..(219): CAT_CLOBU, Type A
MNFNLIDINHWSRKPYFEHYLNNVKCTYSMTANIEITDLLYEIKLKNIKFYPTLIYMIATVVNNHKEFRICFDHKGSLGYWDSMNPSYTIFHKENETFSSIWTEYNKSFLRFYSDYLDDIKNYGNIMKFTPKSNEPDNTFSVSSIPWVSFTGFNLNVYNEGTYLIPIFTAGKYFKQENKIFIPISIQVHHAICDGYHASRFINEMQELAFSFQEWLENK

SEQ ID NO: 14 - protein, 219 aa, Clostridium perfringens
  SITE (1)..(219): CAT1_CLOPF, Type A
MKFNLIDIEDWNRKPYFEHYLNAVRCTYSMTANIEITGLLREIKLKGLKLYPTLIYIITTVVNRHKEFRTCFDQKGKLGYWDSMNPSYTVFHKDNETFSSIWTEYDENFPRFYYNYLEDIRNYSDVLNFMPKTGEPANTINVSSIPWVNFTGFNLNIYNDATYLIPIFTLGKYFQQDNKILLPMSVQVHHAVCDGYHISRFFNEAQELASNYETWLGEK

SEQ ID NO: 15 - protein, 212 aa, Clostridioides difficile
  SITE (1)..(212): CAT_CLODI, Type A
MVFEKIDKNSWNRKEYFDHYFASVPCTYSMTVKVDITQIKEKGMKLYPAMLYYIAMIVNRHSEFRTAINQDGELGIYDEMIPSYTIFHNDTETFSSLWTECKSDFKSFLADYESDTQRYGNNHRMEGKPNAPENIFNVSMIPWSTFDGFNLNLQKGYDYLIPIFTMGKIIKKDNKIILPLAIQVHHAVCDGFHICRFVNELQELIIVTQVCL

SEQ ID NO: 16 - protein, 207 aa, Campylobacter coli
  SITE (1)..(207): CAT_CAMCO, Type A
MQFTKIDINNWTRKEYFDHYFGNTPCTYSMTVKLDISKLKKDGKKLYPTLLYGVTTIINRHEEFRTALDENGQVGVFSEMLPCYTVFHKETETFSSIWTEFTADYTEFLQNYQKDIDAFGERMGMSAKPNPPENTFPVSMIPWTSFEGFNLNLKKGYDYLLPIFTFGKYYEEGGKYYIPLSIQVHHAVCDGFHVCRFLDELQDLLNK

SEQ ID NO: 17 - protein, 216 aa, Vibrio anguillarum
  SITE (1)..(216): CAT_VIBAN, Type A
MEFRLVDLKTWKRKEYFTHYFESVPCTYSMTVKLDITTIKTGKAKLYPALLYAVSTVVNRHEEFRMTVDDEGQIGIFSEMMPCYTIFQKDTEMFSNIWTEYIGDYTEFCKQYEKDMQQYGENKGMMAKPNPPVNTFPVSMIPWTTFEGFNLNLQKGYGYLLPIFTFGRYYEENGKYWIPLSIQVHHAVCDGFHTCRFINELQDVIQSLQNHGGDEE

SEQ ID NO: 18 - protein, 220 aa, Streptomyces acrimycini
  SITE (1)..(220): CAT_STRAC, Type A
MDAPIPTPAPIDLDTWPRRQHFDHYRRRVPCTYAMTVEVDVTAFAAALRRSPRKSYLAQVWALATVVNRHEEFRMCLNSSGDPAVWPVVHPAFTVFNPERETFACLWAPYDPDFGTFHDTAAPLLAEHSRATDFFPQGNPPPNAFDVSSLPWVSFTGFTLDIRDGWDHLAPIFTLGRYTERDTRLLLPLSVQIHHAAADGFHTARLTNELQTLLADPAWL

SEQ ID NO: 19 - protein, 213 aa, Escherichia coli
  SITE (1)..(213): CAT2_ECOLX, Type A
MNFTRIDLNTWNRREHFALYRQQIKCGFSLTTKLDITALRTALAETGYKFYPLMIYLISRAVNQFPEFRMALKDNELIYWDQSDPVFTVFHKETETFSALSCRYFPDLSEFMAGYNAVTAEYQHDTRLFPQGNLPENHLNISSLPWVSFDGFNLNITGNDDYFAPVFTMAKFQQEGDRVLLPVSVQVHHAVCDGFHAARFINTLQLMCDNILK

SEQ ID NO: 20 - protein, 213 aa, Haemophilus influenzae
  SITE (1)..(213): CAT2_HAEIF, Type A
MNFTRIDLNTWNRREHFALYRQQIKCGFSLTTKLDITAFRTALAETDYKFYPVMIYLISRVVNQFPEFRMAMKDNALIYWDQTDPVFTVFHKETETFSALFCRYCPDISEFMAGYNAVMAEYQHNTALFPQGALPENHLNISSLPWVSFDGFNLNITGNDDYFAPVFTMAKFQQEDNRVLLPVSVQVHHAVCDGFHAARFINTLQMMCDNILK

SEQ ID NO: 21 - protein, 213 aa, Escherichia coli
  SITE (1)..(213): CAT3_ECOLX, Type A
MNYTKFDVKNWVRREHFEFYRHRLPCGFSLTSKIDITTLKKSLDDSAYKFYPVMIYLIAQAVNQFDELRMAIKDDELIVWDSVDPQFTVFHQETETFSALSCPYSSDIDQFMVNYLSVMERYKSDTKLFPQGVTPENHLNISALPWVNFDSFNLNVANFTDYFAPIITMAKYQQEGDRLLLPLSVQVHHAVCDGFHVARFINRLQELCNSKLK

SEQ ID NO: 22 - protein, 217 aa, Proteus mirabilis
  SITE (1)..(217): CAT_PROMI, Type A
MDTKRVGILVVDLSQWGRKEHFEAFQSFAQCTFSQTVQLDITSLLKTVKQNGYKFYPTFIYIISLLVNKHAEFRMAMKDGELVIWDSVNPGYNIFHEQTETFSSLWSYYHKDINRFLKTYSEDIAQYGDDLAYFPKEFIENMFFVSANPWVSFTSFNLNMANINNFFAPVFTIGKYYTQGDKVLMPLAIQVHHAVCDGFHVGRLLNEIQQYCDEGCK

SEQ ID NO: 23 - protein, 219 aa, Escherichia coli
  SITE (1)..(219): CAT1_ECOLX, Type A
MEKKITGYTTVDISQWHRKEHFEAFQSVAQCTYNQTVQLDITAFLKTVKKNKHKFYPAFIHILARLMNAHPEFRMAMKDGELVIWDSVHPCYTVFHEQTETFSSLWSEYHDDFRQFLHIYSQDVACYGENLAYFPKGFIENMFFVSANPWVSFTSFDLNVANMDNFFAPVFTMGKYYTQGDKVLMPLAIQVHHAVCDGFHVGRMLNELQQYCDEWQGGA

SEQ ID NO: 24 - protein, 219 aa, Klebsiella sp.
  SITE (1)..(219): CAT_KLESP, Type A
MEKKITGYTTVDISQWHRKEHFEAFQSVAQCTYNQTVQLDITAFLKTVKKNKHKFYPAFIHILARLMNAHPEFRMAMKDGELVIWDSVHPCYTVFHEQTETFSSLWSEYHDDFRQFLHIYSQDVACYGENLAYFPKGFIENMFFVSANPWVSFTSFDLNVAAMDNFFAPVFTMGKYYTQGDKVLMPLAIQVHHAVCDGFHVGRMLNELQQYCDEWQGGA

SEQ ID NO: 25 - protein, 212 aa, Pseudomonas aeruginosa
  SITE (1)..(212): CAT4_PSEAE, Type B
MGNYFESPFRGKLLSEQVSNPNIRVGRYSYYSGYYHGHSFDDCARYLMPDRDDVDKLVIGSFCSIGSGAAFIMAGNQGHRAEWASTFPFHFMHEEPVFAGAVNGYQPAGDTLIGHDVWIGTEAMFMPGVRVGHGAIIGSRALVTGDVEPYAIVGGNPARTIRKRFSDGDIQNLLEMAWWDWPLADIEAAMPLLCTGDIPALYRHWKQRQATA

SEQ ID NO: 26 - protein, 210 aa, Escherichia coli
  SITE (1)..(210): CAT4_ECOLX, Type B
MTNYFESPFKGKLLTEQVKNPNIKVGRYSYYSGYYHGHSFDDCARYLLPDRDDVDQLIIGSFCSIGSGAAFIMAGNQGHRYDWVSSFPFFYMNEEPAFAKSVDAFQRAGDTVIGSDVWIGSEAMIMPGIKIGHGAVIGSRALVAKDVEPYTIVGGNPAKSIRKRFSEEEISMLLDMAWWDWPLEQIKEAMPFLCSSGIASLYRRWQGTSA

SEQ ID NO: 27 - protein, 210 aa, Klebsiella aerogenes
  SITE (1)..(210): CAT4_KLEAE, Type B
MTNYFDSPFKGKLLSEQVKNPNIKVGRYSYYSGYYHGHSFDDCARYLFPDRDDVDKLIIGSFCSIGSGASFIMAGNQGHRYDWASSFPFFYMQEEPAFSSALDAFQKAGNTVIGNDVWIGSEAMVMPGIKIGHGAVIGSRSLVTKDVEPYAIVGGNPAKKIKKRFTDEEISLLLEMEWWNWSLEKIKAAMPMLCSSNIVGLHKYWLEFAV

SEQ ID NO: 28 - protein, 210 aa, Morganella morganii
  SITE (1)..(210): CAT4_MORMO, Type B
MKNYFDSPFKGELLSEQVKNPNIKVGRYSYYSGYYHGHSFDECARYLHPDRDDVDKLIIGSFCSIGSGASFIMAGNQGHRHDWASSFPFFYMQEEPAFSSALDAFQRAGDTAIGNDVWIGSEAMIMPGIKIGDGAVIGSRSLVTKDVVPYAIIGGSPAKQIKKRFSDEEISLLMEMEWWNWPLDKIKTAMPLLCSSNIFGLHKYWREFVV